(19) United States
(12) Patent Application Publication
(10) Pub. No.: US 2005/0154534 A1
(43) Pub. Date: Jul. 14, 2005

(54) Automated media optimization technology

(75) Inventors: Perry D. Haaland, Chapel Hill, NC (US); Bryce N. Chaney, Durham, NC (US); Stacy D. Holdread, Sparks, MD (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(73) Assignee: Becton, Dickinson and Company

(21) Appl. No.: 10/755,684

(22) Filed: Jan. 13, 2004

(51) Int. Cl. G06F 19/00; G01N 33/48; G01N 33/50

(52) U.S. Cl. 702/19

(57) ABSTRACT

An automated system and method for identifying agents capable of eliciting a phenotypic change in a cell-type. The method includes the steps of providing a statistical design including generic factor names, factor levels and experimental runs, and utilizing a software program to generate a computer representation of the statistical design by automatically mapping the identities of the agents to the generic factor names, concentrations or amounts of the agents to the factor levels, and the locations of the receptacles to the experimental runs. The method also includes placing different mixtures of single agents, such as peptones, into receptacles in the array based on the computer representation of the statistical design, contacting the placed mixtures with cells, acquiring experimental data from the contacted cells, and utilizing a processor including an algorithm for comparing the acquired data with the statistical design to identify peptone combinations and concentrations that optimize cell culture conditions.

### CROSS-REFERENCE TO RELATED APPLICATIONS

Related subject matter is disclosed in U.S. Patent Application of Heidaran et al., entitled “Computer Software And Algorithms For Systems Biologically Linked To Cellular Phenotype”, Ser. No. 10/662,713, filed on Sep. 15, 2003, the entire content of which is incorporated herein by reference.

Additional related subject matter is disclosed in U.S. patent application Ser. No. 09/359,260, entitled “Methods, Apparatus And Computer Program Products For Formulating Culture Media”, filed Jul. 22, 1999, in U.S. patent application Ser. No. 10/662,640, entitled “High Throughput Method To Identify Ligands For Cell Attachment”, filed Sep. 15, 2003, in U.S. patent application Ser. No. 09/608,892, entitled “Peptides For Use In Culture Media”, filed Jun. 30, 2003, and in U.S. patent application Ser. No. 10/260,737, entitled Methods And Devices For The Integrated Discovery Of Cell Culture Environments”, filed Sep. 30, 2002, the entire content of each is incorporated herein by reference

### BACKGROUND OF THE INVENTION

1. Field of the Invention

The present invention relates generally to the field of high throughput screening methods. The present invention relates to a computer-implemented screening system and method that can be used to identify agent mixtures that elicit a desired response and specifically, identify the best set and/or subset of two or three peptones that optimizes cell culture conditions based upon a variety of responses such as antibody secretion, cell number and time to peak antibody secretion.

2. Description of the Related Art

For cells to be used in therapies to treat or cure diseases in humans, it is desirable to control cell fate, e.g., cell survival, proliferation and differentiation, when cells are maintained in culture in vitro. It is therefore necessary to control cell surface receptor interaction with ligands. For example, in order to gain control over interactions between a cell and ligands present on the in vitro culture substrate, a suitable culture substrate, such as polystyrene, can be coated with a polymer which does not allow for cell attachment even when serum proteins are used in the culture media. This coating thus eliminates the uncontrolled and arbitrary adsorption of the serum proteins. Biologically active ligands suitable to interact with cell surface receptors can then be immobilized on this coating while maintaining the biological activity of the ligands. This concept is well known to those skilled in the art. For example, it is known to use hyaluronic acid or algenic acid as a surface coating upon which the cell adhesion ligands can be immobilized using chemistries resulting in stable covalent bonds between the coating and the cell adhesion ligands. This prevents the cell adhesion ligand from being solubilized and leaving the surface. Moreover, the coating itself does not support cell adhesion. This is further described in a copending, commonly owned U.S. patent application Ser. No. 10/259,797, filed on Sep. 30, 2002, the entire content of which is incorporated herein by reference.

Additionally, it is probable that specific mixtures of agents are required in order to achieve a desired cell fate. A great number of growth effector molecules are known. These include growth factors, hormones, peptides, small molecules and extracellular binding molecules. However, it can thus be a tedious task to find the right growth effector or growth effector combinations to achieve a desired cell fate for a given cell type.

Accordingly, a need exists for higher throughput system and methods to identify agents useful to achieve a desired cell fate for a given cell type. This is of particular interest for cells that do not survive or only survive by drastically altering their differentiation state in conventional cell culture systems, a prime example being primary mammalian cells. In particular, there is a need in the art for a computer-implemented, statistically designed experimental method and a system for implementation to systematically explore the interactions between mixtures of factors that are required in order to achieve a desired cell fate. Preferably, the higher throughput system and method would include starting from a list of several possible agents, such as peptones, and implement an optimization strategy to identify the best subset of two or three peptones that optimizes cell culture conditions based upon a variety of responses. The responses can include antibody secretion, cell number and peak antibody secretion periods.

### SUMMARY OF THE INVENTION

Accordingly, an object of the present invention is to provide an automated system and method for identifying agents that cause a phenotypic change in a cell. The method includes providing receptacles in an array and providing a statistical design including generic factor names, factor levels and experimental runs. The method further includes placing different mixtures of single agents into select ones of the receptacles according to a computer representation of the statistical design and utilizing a software program to generate the computer representation of the design. The software automatically maps the identities of the agents to the generic factor names, maps the concentration or amounts of the agents to the factor levels and maps the locations of the receptacles within the array to the experimental runs. Once the different mixtures have been correctly placed into receptacles in accordance with the computer representation of the design, the placed mixtures are contacted with whole cells that are capable of changing their phenotype.

Another object of the present invention includes providing a method to acquire data indicative of a phenotypic change in the contacted cells and utilizing a processor including an algorithm for comparing the acquired data with the statistical design to identify which of the agent mixtures and/or which single agents are effective in causing the phenotypic change in the contacted cells. The method further includes storing the statistical design, the identities of the agents, the computer representation of the design, the acquired experimental data and the results of the algorithm comparison in one or more databases.

Yet another object of the present invention includes providing a system for implementing the method just described, and includes an array of receptacles, selective ones of which are for receiving (i) different mixtures of single agents and (ii) fluid including cells. The system also includes a statistical design including generic factor names, factor levels, and experimental runs, and a software program for generating a computer representation of the design. The software program automatically maps the identities of the agents to the generic factor names, maps the concentration of or amounts of the agents to the factor levels and maps the locations of the receptacles within the array to the experimental runs. The system also includes acquired experimental data indicative of the phenotypic change in the cells, and a processor including an algorithm for comparing the experimental data with the statistical design to identify the mixtures and/or single agents which are effective in causing the phenotypic change in the cells. Further included in the system are one or more databases for storing the statistical design, the agent identities, the computer representation of the design, the acquired experimental data and the results of the algorithm comparison.

Still another object of the present invention is to apply an automated media optimization technology that enables users to optimize media components (e.g. factors) using the MPM/CATSBA software and robotic liquid-handling platforms. Using specific factors, the MPM/CATSBA software automatically creates statistically designed experiments in a multi-well plate format, and generates the necessary files to prepare the correct experimental conditions using a robotic-liquid-handling platform (e.g. the Biomek FX, Biomek 2000, Tecan Genesis, or any similar platforms). The software and the database it resides on are used to automatically categorize and analyze numerous formats of data (e.g. fluorescence, absorbance, cell counts, and so forth). The software user can perform all relevant statistical analyses in an automated fashion and all relevant reports are automatically generated and stored within the database. After all relevant statistical analyses are performed, the user has the ability to combine results from multiple experiments for a meta-analysis and data mining.

### BRIEF DESCRIPTION OF THE DRAWINGS

These and other objects, advantages and novel features of the invention will be more readily appreciated from the following detailed description when read in conjunction with the accompanying drawings, in which:

FIGS. 1A and 1B are flow diagrams of an example automated process in accordance with a embodiment of the present invention by which best mixtures and/or best agents capable of eliciting a phenotypic change in a cell can be identified in high throughput fashion;

FIG. 2 is a flow diagram of an example process in accordance with an embodiment of the present invention by which a biological model can be created and/or revised using the information derived from the process in FIGS. 1A and 1B;

FIG. 3 illustrates an example model of exemplary cellular pathways hypothesized for the action of an agent mixture (M) on a biological system;

FIG. 4 is a block diagram of an example of a computer system which can be used to carry out a method of FIGS. 1-3 in accordance with an embodiment of the present invention;

FIG. 5 is a block diagram which illustrates an example of the components of the system in accordance with an embodiment of the present invention;

FIG. 6 is a schematic representation of an example test well in accordance with an embodiment of the present invention;

FIG. 7 is a schematic representation of an example 96-well plate layout comprising different mixtures of single agents wherein the layout is created using a statistical design in which generic factors in the design represent single agents and are combined to form the different mixtures in accordance with an embodiment of the present invention;

FIGS. 8A and 8B are block diagram representations of an example scenario that can be used in developing the statistical design of the method in accordance with an embodiment of the present invention;

FIGS. 9A and 9B are block diagram representations of another example scenario that can be used in developing the statistical design of the method in accordance with an embodiment of the present invention;

FIG. 10 is an example spreadsheet computer representation of a mixture design having a layout for a 96-well plate developed using the scenario of FIGS. 9A and 9B in accordance with an embodiment of the present invention, wherein the total fluid volume in a well is divided up based on the number of factors present;

FIG. 11 shows an example 96-well plate layout based on a statistical design of the spreadsheet in FIG. 10 in accordance with an embodiment of the present invention;

FIG. 12 is an example fluorescent microscope image of fluorescently labeled cells attached to the wells of the 96-well plate with the layout shown in FIG. 11 in accordance with an embodiment of the present invention;

FIG. 13 is an example graph of the nuclei count vs. well number obtained following an analysis of the microscope image in FIG. 12 in accordance with an embodiment of the present invention;

FIG. 14 is an example graph of Ln (cell count-no serum+1) vs. deviation from the reference blend obtained using a mixture-model analysis of information from FIGS. 9-13 in accordance with an embodiment of the present invention;

FIG. 15 is an example graph of Ln (cell count-10% serum+1) vs. deviation from the reference blend obtained using a mixture-model analysis of information from FIGS. 9-13 in accordance with an embodiment of the present invention;

FIGS. 16a through 16d are example spreadsheets showing a Plackett-Burman statistical design for the layout of a 96-well plate in accordance with an embodiment of the present invention;

FIG. 17 shows an example of the identity of the factors in an example statistical design in FIG. 16 in accordance with an embodiment of the present invention;

FIG. 18 is an example graph showing secretion and cell proliferation over time for a first substance in accordance with an embodiment of the present invention;

FIG. 19 is an example graph showing secretion and cell proliferation over time for a second substance in accordance with an embodiment of the present invention; and

FIG. 20 is an example graph showing combined secretion and cell proliferation over time for both the first and second substance in accordance with an embodiment of the present invention.

### DETAILED DESCRIPTION OF THE PREFERRED EMBODIMENT

As defined herein, “agents” are growth effector molecules that bind to cells and regulate the survival, differentiation, proliferation or maturation of target cells or tissue. Examples of suitable agents for use in the embodiments of the present invention include peptones, growth factors, extracellular matrix molecules, peptides, hormones and cytokines which can either be in solution or bound to a culture surface, such as a well surface, scaffold surface, bead surface, and so forth.

The term “agent-immobilizing material” is defined herein as a biocompatible polymer that can serve as a link between the culture surface and an agent.

As defined herein, the term “immobilize,” “immobilized,” and the like is to render an agent, i.e., growth effector molecules, immobile on a culture surface, such as a well surface or the surface of a scaffold contained within a well. This term is intended to encompass passive adsorption of the agents to the culture surface, as well as direct or indirect covalent attachment of the agents to the culture surface.

“Factors” are the names of the variables in the experiment, and represent the elements that the experiment changes from one trial or run (e.g., one well) to the next. In the embodiments of the present invention, “factor” is a generic name for a single agent or mixture of single agents. Factors are combined according to a statistical design to form different mixtures in the experiment.

“Statistical Design”, as defined herein, is an experimental design that assists the user in finding a combination of adjustable variables (i.e., factors) to produce the best experimental outcome, dramatically reducing the number of experiments needed to achieve that objective. In the embodiments of the present invention, a suitable statistical design is generated using generic factor names which represent the agents being tested. The design includes factor levels that can be the amounts and/or concentrations of the factors or that can be converted to the actual amounts and/or concentrations of the factors. The design also includes experimental runs which are numbered. Experimental runs specify the combinations of factors and the levels thereof to test, and each can correspond to a single well on a multiwell plate. The experimental runs can be mapped to wells on a generic multiwell plate.

As used herein, the terms “pre-treatment” and “pre-treated” refers to the addition to a surface or other substrate of functional groups which are chemically involved in the covalent bond subsequently formed with the agent-immobilizing material (i.e., a biocompatible polymer). For example, a surface of a microtitre well can be subjected to amino-plasma treatment to create an amine-rich surface onto which the agent-immobilizing material may be coupled.

The term “array,” “receptacle array,” and the like as defined herein is a plurality of unique containers, such as tubes or wells, which are placed in an orderly arrangement, such as rows and columns.

The term “phenotypic”, “phenotypic change”, and the like as defined herein includes the observable physical or biochemical characteristics of an organism, as determined by both genetic makeup and environmental influences. This includes the expression of a specific trait, such as antibody secretion, cell number and time to peak antibody secretion, based on genetic and environmental influences.

As described above, it is likely that mixtures of single agents are required in order to achieve a desired cell fate. For example, growth effector molecules that bind to cell surface receptors and regulate the survival, differentiation, proliferation or maturation of these cells include growth factors, extracellular matrix molecules, peptides, hormones and cytokines, of which there are many examples. It can therefore be a tedious task to find the right growth effector or growth effector combinations to place in contact with the cell to achieve a desired cell fate.

The embodiments of the present invention solve a need in the art by providing a high throughput, computer-implemented method to identify optimal agents for a given cell type. Specifically, the system and method described in greater detail below, examines a list of several possible agents, such as peptones, and identifies the best subset of two or three peptones that optimizes cell culture conditions based on a variety of responses, such as antibody secretion, cell number, and time to peak antibody secretion.

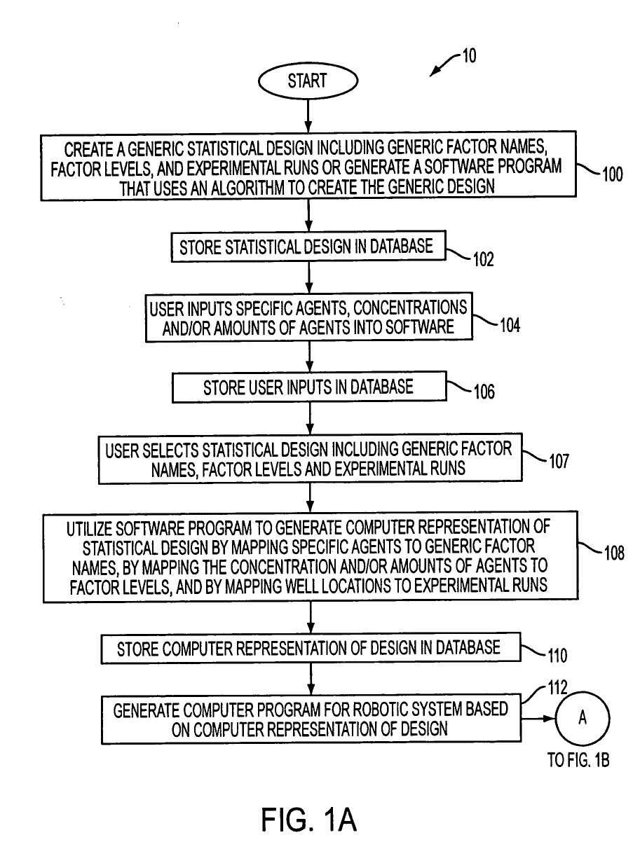

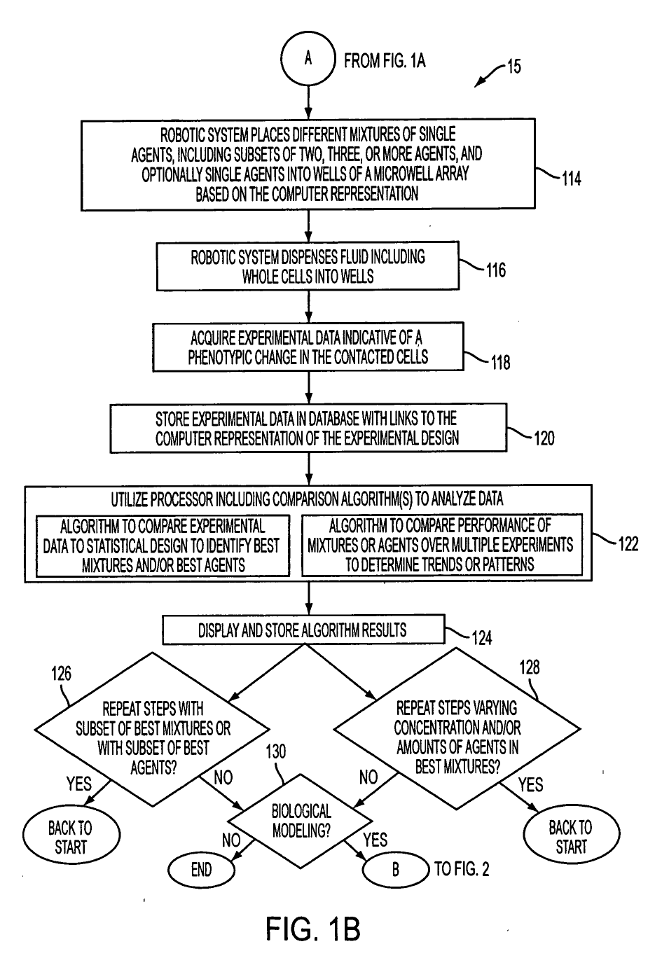

FIGS. 1A and 1B are flow diagrams of an example process in accordance with an embodiment of the present invention by which, unique mixtures and/or single agents can be identified that are capable of eliciting a change in the phenotype of a cell. In the embodiment described below, the format used is that of a microwell array. In general, such arrays are well suited to automation, since automatic pipetters and plate readers are readily available.

In a first step of flow diagram 10, a user at block 100 either creates an experimental design using a commercially available software such as JMP™, available from SAS Institute of Cary, N.C., or generates a statistical design based on an algorithm that is already included in the software of the system. The design at block 100 includes generic factor names, factor levels, experimental runs, and a mapping of experimental runs to a generic microwell array. The statistical design is stored in a database at block 102. The user then inputs the specific agents at block 104, as well as the concentrations and/or amounts of the specific agents into the software. The user inputs are stored in a database at block 106.

At block 107, the user can select a specific statistical design. Subsequently, at block 108 a software program is utilized in order to generate a computer representation of the specific statistical design. The computer representation of the design can be a spreadsheet which can translate for example, into a 96-well layout. In particular, the software program used to generate the computer representation maps the names of the specific agents to the generic factor names in the design, maps the concentration and/or amounts of the agents to the factor levels in the design, creates experimental runs based on the specific agents and the concentrations and/or amounts, and maps the well locations to the experimental runs on a specific microwell array. The computer representation is then stored in a database at block 110.

At block 112, a computer program is generated for a robotic system based on the computer representation of the design. At block 114 as shown in flow diagram 15 of FIG. 1B, the robotic system dispenses the agents into the wells according to the computer representation of the design so as to generate different mixtures in select ones of the wells in the microwell array, including subset mixtures of two and three agents. Optimally, the robotic system can dispense single agents into others of the wells in the microwell array. In addition to reagent addition, withdrawal and wash steps can be performed by the robotic system. Alternatively, some or all of these steps can be performed manually. The agents can be tethered covalently to the well or other culture surface via a biocompatible polymer such as algenic acid or hyaluronic acid or may be present in solution.

Once the agents have been placed into the wells correctly, the robotic system at block 116 dispenses fluid including whole cells into the wells of the microwell array. At block 118 experimental data is acquired which would be indicative of a change in the phenotype of a cell. The acquired data is stored in a database at block 120 so that the experimental data is linked to the computer representation of the design. Then at block 122, a processor is utilized which includes an algorithm to compare the stored experimental data to the stored statistical design to identify the best mixtures and/or best agents, and in particular, which subset of two or three elicited the desired biological response (i.e., elicited a phenotypic change in the cells). Optionally, another algorithm can be used to compare the performance of mixtures of agents or single agents over multiple experiments to determine trends or patterns. In either case, the results of the algorithm comparisons can be stored in a database and displayed to a user at block 124, and can be periodically updated.

The databases shown in FIGS. 1A and 1B can be a single integrated or federated database. At block 126, if desired, the steps of the process can be repeated with a subset of the best mixtures or a subset of the best agents. Moreover, if desired, the steps can be repeated with a combined subset of best agents and a subset of agents from the best mixtures (not shown). Furthermore, at block 128 the steps of the process can be repeated, varying the concentration and/or amounts of the agents in the best mixtures. Additionally, information acquired from the algorithm comparisons at block 122 can optimally be used to create or revise a biological model at block 130.

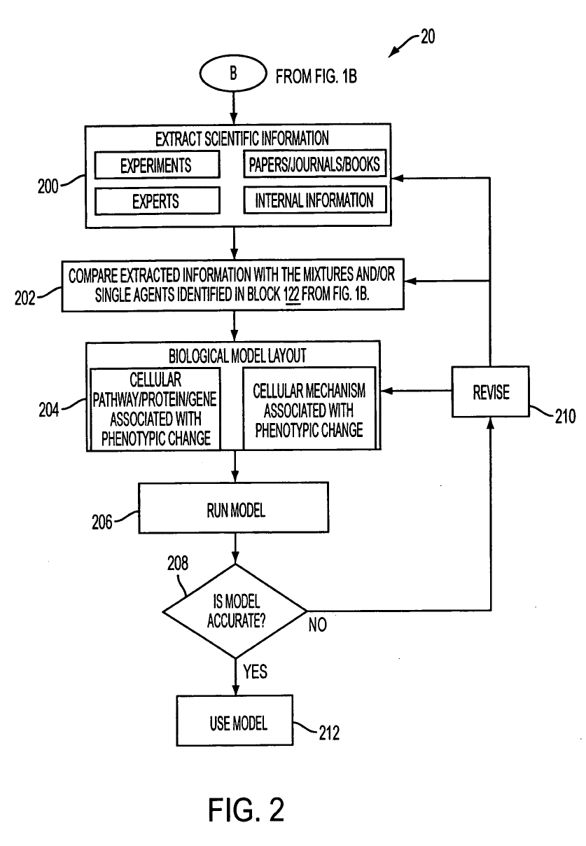

Referring now to FIG. 2, a flow diagram is presented of an example process in accordance with an embodiment of the present invention by which a biological model can be created or an existing model can be revised using the information from blocks 122 and 124 of FIG. 1B. In a first step of flow diagram 20, scientific information is collected from a variety of sources, such as papers, journals, books, experts, experiments, internal information, and so forth, at block 200. Scientific information can include, but is not limited to, gene expression data, protein expression data, cellular phenotype data, signal transduction data, data on cellular pathways, and combinations thereof. Such scientific information can be stored in one or more databases. The information can be computer-extracted, such as via the internet.

The extracted information is compared at block 202 with the agent mixtures and/or single agents identified in block 122 from FIG. 1B. Based on this comparison, a biological model can be developed at block 204 or revised. The biological model can define the biological systems involved in the phenotypic change, and any relevant communication mechanisms between biological systems. For example, at block 204, a specific cellular pathway, protein or gene associated with the phenotypic change in the cell may be identified. In one embodiment, the processor described in FIG. 1B further includes a first application program for calculating the likelihood that a cellular pathway, protein, or gene is involved in changes in cellular phenotype associated with an identified mixture of single agents. The cellular pathway, protein, or gene is determined using the extracted scientific information.

In FIG. 2, the biological model can be a computer-executable model, which is run at block 206, checked for accuracy at block 208 and revised at block 210, if necessary. Once the model is determined to be accurate, it can be used at block 212. An example of a computer-executable model of a biological system is described in U.S. Pat. No. 5,808,918, to Fink et al., the entire content of which is incorporated herein by reference. Desirably, the model would be able to support computation, updating, comparison and visualization.

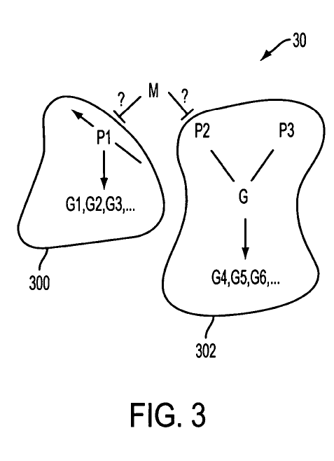

FIG. 3 illustrates an example model of cellular pathways hypothesized for the action of an agent mixture (M) on a biological system. In the model 30 of FIG. 3, agent mixture (M) acts on a cell by interacting with hypothetical biological pathways 300 and 302. The arcs between mixture (M) and these pathways represent possible action of mixture (M) on these pathways. The entire action of mixture (M) on the cells is assumed to be expressible as a combination of mixture (M) actions on one or more of these two pathways. As used herein, a cellular pathway is generally understood to be a collection of cellular constituents related in that each cellular constituent of the collection is influenced according to some biological mechanism by one or more other cellular constituents in the collection. The cellular constituents making up a particular cellular pathway can be drawn from any aspect of the biological state of a cell, for example, from the transcriptional state, or the translational state, or the activity state, or mixed aspects of a biological state.

Cellular constituents of a cellular pathway can include mRNA levels, protein abundances, protein activities, degree of protein or nucleic acid modification (e.g., phosphorylation or methylation), combinations of these types of cellular constituents, and so forth. Each cellular constituent is influenced by at least one other cellular constituent in the collection by some biological mechanism. The influence, whether direct or indirect, of one cellular constituent on another is presented as an arc between the two cellular constituents and the entire pathway is presented as a network of arcs linking the cellular constituents to the pathway.

In FIG. 3, biological pathway 300 includes protein P1 (i.e., for example, either the abundance or activity of P1) and genes G1, G2, and G3 (i.e., for example, the transcribed mRNA levels). Biological pathway 300 further includes the influence, whether direct or indirect, of protein P1 on these three genes represented as the arc leading from P1 to these three genes. This influence might arise, for example, because protein P1 can bind to promoters of these genes and increase the abundance of their transcripts. Also shown in FIG. 3 is cellular pathway 302. In this pathway, proteins P2 and P3 both directly affect gene G. In turn, gene G, perhaps indirectly, affects genes G4, G5 and G6.

In order to ascertain certain pathways, proteins, or genes of particular interest, aspects of the biological state of the cell, for example, the transcriptional state, the translational state, or the activity state, can be measured in the presence of a mixture of single agents identified as eliciting a phenotypic change in the cell. This corresponds with block 122 of FIG. 1B. In one example, cellular pathways or mechanisms can be identified by identifying genes and/or proteins expressed by the cells in the presence of the identified mixture of single agents. In another example, cellular pathways or mechanisms can be identified by identifying receptors on the cells which are activated in the presence of the identified mixture of single agents.

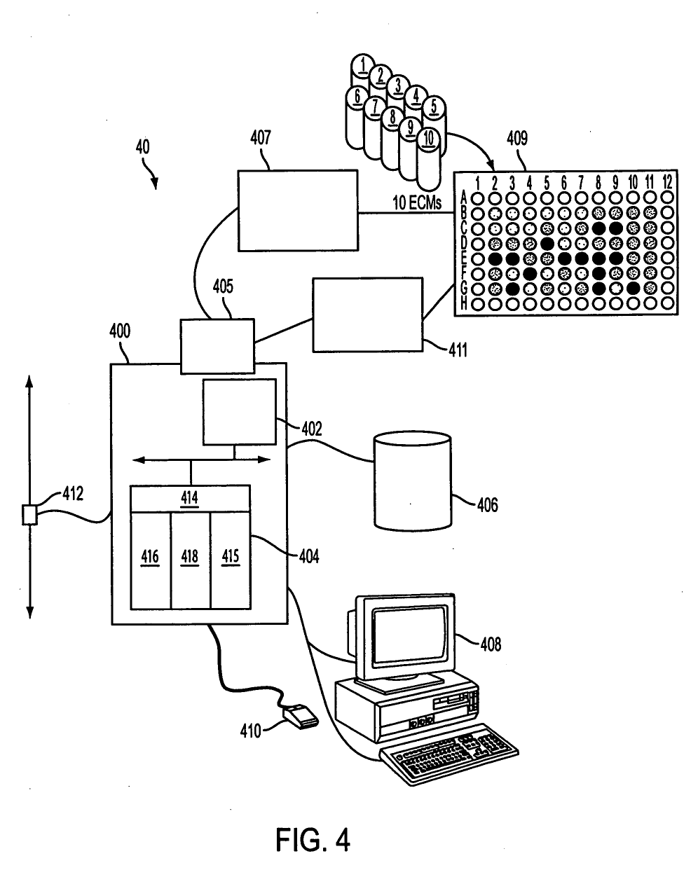

FIG. 4 illustrates an example computer system suitable for the implementation of the methods described in FIGS. 1 through 3. In the block diagram 40, computer system 400 is shown as including internal components and being linked to external components in the embodiment shown. The internal components include processor 402 interconnected with a main memory 404. In one example, computer system 400 can be an Intel Pentium®-based processor of 200 Mhz or greater clock rate and with 32 Mb or more of main memory. External components can include one or more hard disks 406, which are typically packaged together with the processor and memory. The external components further include interface board 405, microwell plate reader 407 and microwell array 409, which together allow experimental data to be communicated to computer system 400. The external components further include robotic system 411, which places experimental factors such as the ten extracellular matrix proteins (ECM) shown in FIG. 4 into the receptacles of microwell array 409 in accordance with a statistical design selected by a user.

Other external components can include user interface device 408, which can be a monitor and keyboard, together with pointing device 410, which can be a “mouse”, or other graphic input devices (not illustrated). Typically, the computer system 400 is also linked to network link 412 which can be part of an Ethernet link to other local computer systems, remote computer systems, or the Internet. This network link 412 allows computer system 400 to share data and processing tasks with other computer systems.

Loaded into the memory 404 are several software components which are both standard and well known to those skilled in the art, and components that are particular to the embodiment of the present invention. These software components collectively result in the computer 400 system to function according to the methods of at least one embodiment of the present invention. The software components are typically stored on hard disks 406. Software component 414 represents the operating system, which is responsible for managing computer system 400 and the network interconnections. An example of a suitable operating system is Windows 98, or Windows NT. Software component 415 is provided for analyzing the image from the microwell plate reader 407, and software component 416 represents common languages and functions conveniently present on system 400 to assist programs implementing the methods which are specific to the embodiment. Languages that can be used to program the analytical methods include Java®, but may also include C, C⁺⁺, Fortran, Visual Basic or other computer languages.

In one example, the method can be programmed in mathematical software packages which allows symbolic entry of equations and high-level specification of processing, including algorithms to be used, thereby freeing a user of the need to procedurally program individual equations or algorithms. Such packages include Matlab, available from Mathworks of Natick, Mass., Mathematica available from Wolfram Research of Champaign, Ill., S-Plus available from Mathsoft of Seattle Wash., MathCAD available from Mathsoft of Cambridge, Mass., and “R” available from the R Foundation. Accordingly, software component 418 represents the methods as programmed in a procedural language or symbolic package.

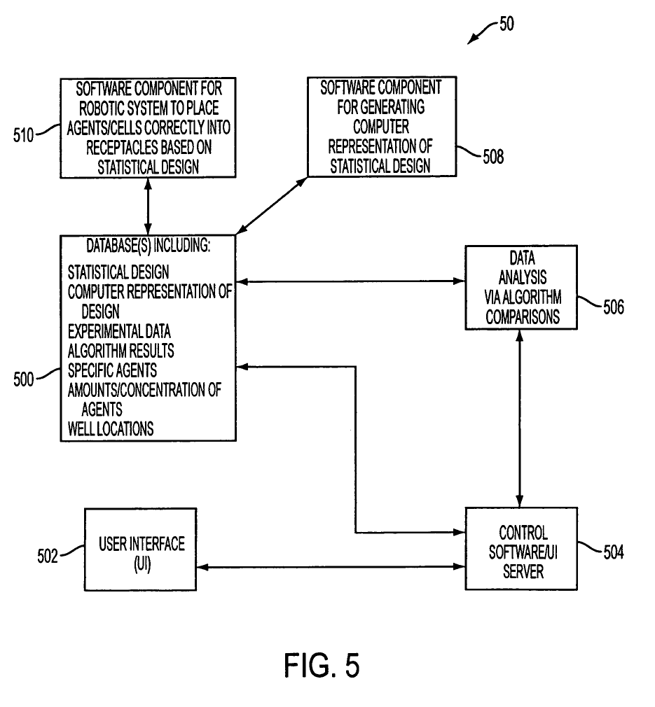

In the system of FIG. 4, a software component 418 further includes several software components which interact with each other as illustrated in block diagram 50 of FIG. 5. Software component 500 represents a database, which is preferably a single integrated or federated database containing data necessary for the operation of computer system 400. Such data preferably includes the statistical design, the computer representation of the statistical design, experimental data, algorithm results, the names of specific agents tested, amounts and/or concentrations of the agents tested, and well locations which are to be used in the experiment.

Software component 502 represents a user interface (UI), which is preferably a graphical user interface (GUI), which is a graphical way to represent the operating system, such as Windows 2000 or X11. User interface 502 provides a user of the computer system 400 with control and input as to the statistical design, specific agents, their concentrations and/or amounts, and, optionally, experimental data. The user interface may also include a means for loading information, such as experimental data from the hard drive 406, from removable media (e.g., CD-Rom), or from a different computer system communicating with the instant system over a network, such as the Internet.

Software component 504 represents the control software, which can be referred to as a UI server, which controls the other software components of the computer system. Software component 506 represents a data reduction and computation component including algorithms which execute the analytic methods. For example, component 506 can include an algorithm for comparing acquired experimental data to the statistical design to identify the best mixtures and/or best agents. The data can be imported into the software and automatically linked to the statistical design so that the data is fully annotated and ready for statistical analysis. Moreover, component 506 can include an algorithm to compare the performance of mixtures or agents over multiple experiments to determine trends or patterns which can be stored and periodically updated if desired. In one embodiment, software component 506 includes a linear regression algorithm. This is a method by which coefficients are estimated for each of the specific agents that are used in the statistical design.

Software component 418 can also include a software component 508 for generating a computer representation of the statistical design, as well as a software component 510 for a robotic system to place agents correctly into the wells of array 409 based on the statistical design stored in database 500. For example, a user can select an option via user interface 502 to generate computer files that can be imported into a robotic sample preparation platform, such as Biomek FX, Biomek 2000, Tecan Genesis, or any similar platform. The computer files can be used to automatically prepare the correct experimental conditions on the microwell array, to culture the cells, and to perform any fluid dispensing, fluid withdrawing or wash steps to carry out assays of phenotype.

The method described above can also be implemented from a customer location that is remote from the actual laboratory where the experiments are being performed. This could involve a web-based interface or the distribution of a thick-client software application to the customer. The level of interaction between the laboratory and the customer could vary. For example, the customer could have complete control of the process or, alternatively, the customer could receive only periodic reports from the laboratory as to the progress in obtaining optimal mixtures of agents.

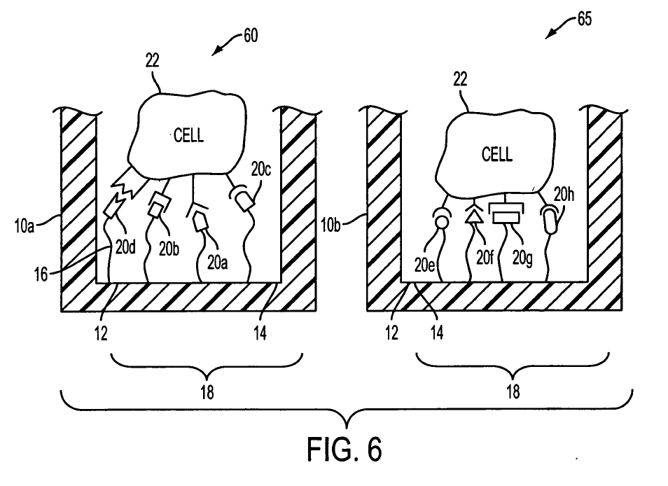

With reference now to FIG. 6, an example test well 60 and 65 is shown, each including receptacles 10 provided in an array (not shown). Receptacles 10 include a surface 12, which can be pre-treated. In one example, surface 12 is amino-plasma treated so as to create an amine-rich surface 14 onto which agent-immobilizing material 16 can be attached. As will be described in further detail below, the agent-immobilizing material 16 is preferably a biocompatible polymer which has been coupled to aminated surface 14.

In the embodiment shown, mixtures 18 of single agents 20, e.g., 20a-d are covalently immobilized to agent-immobilizing material 16. However, some or all of the agents being tested can be in a solution, rather than bound to a culture surface. In one example of the method, mixtures of single agents are covalently immobilized to an agent-immobilizing material on a culture surface, such as the receptacle surface or the surface of a scaffold contained within the receptacle. In yet another example, mixtures of single agents can be passively adsorbed onto a culture surface. Moreover, some or all of the single agents in the mixture can be in solution, and as described above, suitable agents for testing include, but are not limited to, growth factors extracellular matrix molecules, peptides, hormones, and cytokines. Moreover, small molecules, metals, chelators or enzymes can be added as agents to the wells.

Different mixtures 18 of single agents 20 are placed into the receptacles 10 according to a statistical design, which will be described in greater detail below. As shown in FIG. 6, the composition of agents 20a-d in receptacle 10a is different from that in a second receptacle 10b, where the composition comprises single agents 20e-h. It is noted, however, that more than one receptacle can include the same agent. For example, a given agent may have a positive effect on achieving a desired cell fate when surrounded by a certain combination of other agents, and this same agent may have a neutral effect or no effect on achieving a desired cell fate when surrounded by a different combination of agents. Therefore, it would be of benefit to provide an agent in different compositions with other agents to assess these effects.

Referring again to FIG. 6, once agents 20 have been placed as different mixtures into the various receptacles 10 according to a statistical design, these mixtures 18 are contacted with whole cells 22. Agents 20 bind to cells 22 and are capable of producing the desired biological response in the contacted cells. A determination as to the effectiveness of a given mixture of agents or of single agents within the mixture at eliciting the desired response in the cell-type is ascertained based on acquired experimental data. This data can be acquired using methods including, but not limited to, immunocytochemistry analysis, microscopy or functional assays.

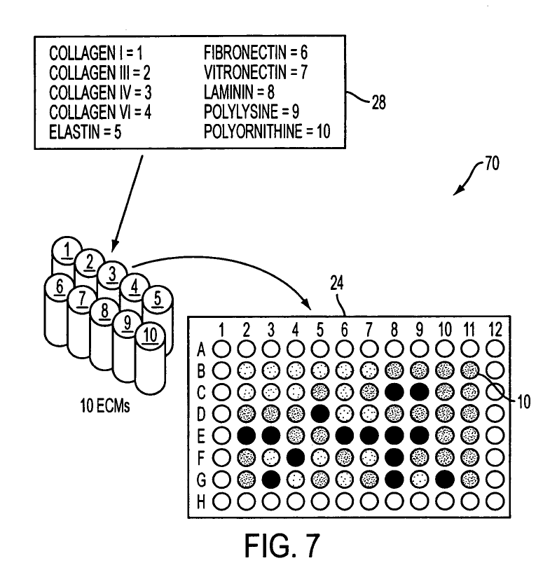

Referring now to FIGS. 7 through 9, aspects of the statistical design will now be described in further detail. Referring in particular to FIG. 7, receptacles 10 are shown in the layout 70 of FIG. 7, and which correspond to a microwell array 24, such as a 96-well plate which is comprised of rows A-H and columns 1-12. As shown in FIG. 7, the identity of single agents 20 or mixtures 18 in FIG. 6 is represented by generic factor names wherein the factors are the variables in the experiment. For example, in the embodiment shown in FIG. 7, generic factors 1-10 are representative of the ten single extracellular matrix proteins indicated in box 28. In this example, generic factor 1 is Collagen I, generic factor 2 is Collagen III, and so forth. Each of these factors can be combined with one or more of the other factors to generate mixtures for the plate layout.

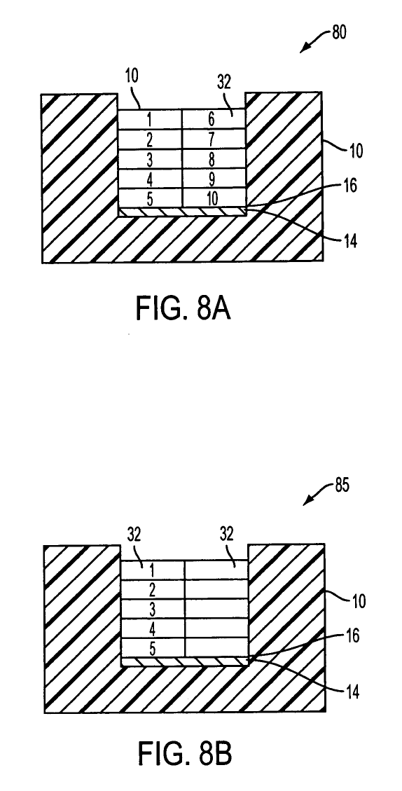

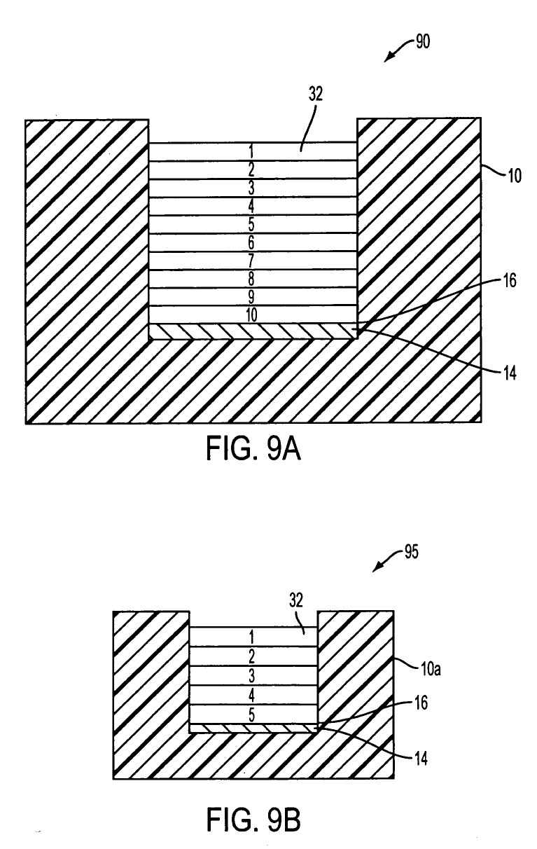

FIGS. 8A, 8B, 9A and 9B will now be described with reference to the embodiment shown in FIG. 7, wherein each of generic factors 1-10 corresponds to a single agent at a given concentration or amount (i.e. factor level).

As shown in FIGS. 8A and 8B, a scenario 80 is presented in which the total fluid volume within receptacle 10 is divided into ten equal volume compartments 32. Each well of a 96-well plate may contain all ten factors (e.g., single agents) or a subset of these factors. As shown in FIG. 8A, in scenario 80 all ten factors are present and all ten factors occupy a fluid compartment 32. The overall factor concentration in well 10 shown in FIG. 8A, is therefore:
[10/10]=[1]
This provides an overall factor concentration that is equivalent to [1] per well.

FIG. 8B represents a different well scenario on the same 96-well plate. In scenario 85, only five out of the ten factors are present. Again, the fluid volume is divided into ten equal compartments 32. In 85, when a factor is present, the fluid compartment is filled with the factor. However, in 85 five out of the ten volume compartments are not filled with a factor, but are rather filled with a “place holder”, such as media. In FIG. 8B, the overall factor concentration equals:
[5/10]=[0.5]

Therefore, the overall factor concentration in the wells shown in FIG. 8B is equivalent to [0.5] per well. The overall factor concentration in 80 is not equivalent to the overall factor concentration in 85. Therefore, the total concentration of the agents in each receptacle can be different. Moreover, in both 80 and 85, the concentration of a single factor is the same between wells. For example, the concentration of factor 1, which can represent a single Collagen I ligand is the same between the well of 80 and the well of 85.

With reference now to FIGS. 9A and 9B, another two scenarios are presented wherein specific consideration is given to the surface chemistry requirements. In particular, in these scenarios the overall density of factor is kept constant from well to well and only the factor composition is allowed to change between wells. In other words, the concentration of a factor can be different from well to well, but each well has the same amount of factor immobilized overall.

As shown in FIGS. 9A and 9B, the total fluid volume present in a given well is divided up based on the number of factors present. Again, for the sake of simplicity, it can be assumed that one factor corresponds to one single agent, although the embodiment shown is not limited to one single agent. As shown in scenario 90 of FIG. 9A, all ten factors are present and the overall factor concentration equals:
[10/10]=[1]
for an overall factor concentration equivalent to [1] per well.

In scenario 95 of FIG. 9B, only five out of the ten factors are present, but the fluid volume 32 of each of these five factors is two times that of the volumes 32 of each of the factors shown in FIG. 9A. Consequently, the overall factor concentration shown in FIG. 9B is the same as that shown in FIG. 9A for an overall factor concentration equivalent to [1] per well.

Therefore, the total concentration of the agents in each receptacle is the same. Based on FIGS. 9A and 9B, it can be seen that whereas the overall factor concentration is constant between the well shown in 9A and the well shown in 9B, the concentration of a single factor can be different between these wells. In particular, with reference to factor 1, which may be representative of Collagen I, the concentration of this single agent in FIG. 9B would be twice that shown in FIG. 9A. Therefore, in yet other examples the concentration of an individual agent can differ between the receptacles. It is noted that each of the scenarios depicted in FIGS. 8 and 9 are feasible and can be used for screening mixtures of single agents.

The methods described above use a format, such as a microwell array, to screen a plurality of different mixtures of agents in parallel for their ability to bind to a given cell-type and elicit a desired response in the cell. The methods include placing different mixtures of agents into selective wells of a multi-well plate according to a statistical design. The methods can further include the optional step of placing single agents into other wells.

The methods also include delivering a fluid sample comprising a cell-type to the wells. After an appropriate incubation time between the cells and the samples in the various wells, evidence of an interaction between the cells and the well components can be detected, either directly or indirectly. For example, data can be acquired using functional assays, immunocytochemistry, or microscopy to measure responses such as antibody secretion, cell number and peak antibody secretion.

Suitable statistical designs for use with the embodiments of the present invention include, but are not limited, to the following: fractional factorial design, D-optimal design, mixture design and Plackett-Burman design. The statistical design can also be a space-filling design based on a coverage criteria, a lattice design, or a latin square design.

As described above, agents can either be bound to a culture surface (e.g., receptacle surface or scaffold surface) or can be in a solution. For example, in one example, the culture surface, which may be pre-treated, is coated with an agent-immobilizing material. The agent-immobilizing material is desirably a biocompatible polymer which does not support cell adhesion and which can serve as a flexible link, or tether between the culture surface and the agents. Examples of suitable polymers include synthetic polymers like polyethylene oxide (PEO), polyvinyl alcohol, polyhydroxylethyl methacrylate, polyacrylamide, and natural polymers such as hyaluronic acid and algenic acid.

Culture surfaces (e.g., well surfaces) are selected from, but not limited to, the following: polystyrenes, polyethylene vinyl acetates, polypropylene, polymethacrylate, polyacrylates, polyethylenes, polyethylene oxide, glass, polysilicates, polycarbonates, polytetrafluoroethylene, fluorocarbons, and nylon. The culture substrates may also wholly or partially include biodegradable materials such as polyanhydrides, polyglycolic acid, polyhydroxy acids such as polylactic acid, polyglycolic acid and polylactic acid-glycolic acid copolymers, polyorthoesters, polyhydroxybutyrate, polyphosphazenes, polypropyl fumurate, and biodegradable polyurethanes.

The culture surfaces can also be pre-treated. For example, cell culture surfaces bearing primary amines can be prepared by plasma discharge treatment of polymers in an ammonia environment. In one example, an agent-immobilizing material can be covalently attached to these aminated surfaces using standard immobilization chemistries as described in copending, commonly owned U.S. patent application Ser. No. 10/259,797, referenced above.

Two processes used commercially to create tissue culture treated polystyrene are atmospheric plasma treatment, also known as corona discharge, and vacuum plasma treatment, each of which is well known to those skilled in the art. Plasmas are highly reactive mixtures of gaseous ions and free radicals. An amino-plasma treatment or oxygen/nitrogen plasma treatment can be used to create an amine-rich surface onto which biocompatible polymers such as hyaluronic acid (HA) or algenic acid (AA) may be coupled through carboxyl-groups using carbodiimide bioconjugate chemistries, as described in U.S. patent application Ser. No. 10/259,797 referenced above. The resulting surfaces will not allow cells to attach, even in the presence of high, e.g., 10-20%, serum protein concentrations present in the cell culture media.

An example of pre-treated tissue culture polystyrene products that can be used to covalently link the agent via the agent-immobilizing material are the PRIMARIA™ tissue culture products available from Becton Dickinson Labware, which are created using oxygen-nitrogen plasma treatment of polystyrene and which result in the incorporation of oxygen-and nitrogen-containing functional groups, such as amino and amide groups.

Agents such as extracellular matrix proteins, peptides, and so forth can be subsequently covalently coupled to the HA or AA surface described above utilizing the amine groups on the proteins/peptides and either the carboxyl groups on the HA or AA, or aldehyde groups created on the HA or AA by oxidation using a substance such as sodium periodate.

In one example, the terminal sugar of human placental hyaluronic acid can be activated by the periodate procedure as described in a publication by E. Junowicz and S. Charm, entitled “The Derivatization of Oxidized Polysaccharides for Protein Immobilization and Affinity Chromatography,” published by Biochimica et. Biophysica Acta, Vol. 428: 157-165 (1976), the entire content of which is incorporated herein by reference. This procedure entails adding sodium or potassium periodate to a solution of hyaluronic acid, thus activating the terminal sugar which can be chemically cross-linked to a free amino group on an agent such as the terminal amino group on an extracellular matrix protein.

In another example, free carboxyl groups on the biocompatible polymer, such as HA or AA, may be chemically cross-linked to a free amino group on the agent using carbodiimide as a cross-linker agent. Still other standard immobilization chemistries are well known to those skilled in the art and can be used to join the culture surfaces to the biocompatible polymers, and to join the biocompatible polymers to the agents. Additional details are provided in a publication by Richard F. Taylor, Ed., entitled “Protein Immobilization: Fundamentals and Applications”, published by M. Dekker, NY, 1991, the entire content of which is incorporated herein by reference, or in copending U.S. patent application Ser. No. 10/259,797, referenced above.

The agents can be tethered to aminated tissue culture surfaces via biocompatible polymers, or can be tethered via biocompatible polymers to carboxylated surfaces or hydroxylated surfaces using standard immobilization chemistries. Examples of attachment agents include cyanogen bromide, succinimide, aldehydes, tosyl chloride, avidin-biotin, photocrosslinkable agents, epoxides and maleimides. Again, it is noted that the agents can be present in a solution and need not be bound to the culture surface.

As described above, the method provides mixtures of agents, which can be bound to a culture surface or can be in solution, contained within selective ones of the receptacles. Moreover, other receptacles may contain a single agent, and the agents may be combined in any desired proportions. The relative amounts of different agents present in the receptacles can be controlled for example, by the concentration of the agents in a composition which is to be dispensed into the receptacles.

Moreover, where the agents are covalently attached via a biocompatible polymer to the receptacle surface, the loading density can be controlled by adjusting the capacity of the biocompatible polymers bound to the culture surface. This can be accomplished by controlling the number of reactive groups on the polymers that can react with the agents, or by controlling the density of the biocompatible polymer molecules on the culture surface. Furthermore, the agents can first be separately linked to the biocompatible polymers (i.e. tethers), and then the “loaded” tethers can be mixed in the desired proportions and attached to the pre-treated substrate.

The agents can be in a solution and/or can be bound to a surface. For example, the agents can be covalently immobilized via biocompatible polymers to a pre-treated tissue culture surface which is desirably amine-rich. Alternatively, the agents can be immobilized to the receptacle surfaces by passively adsorbing the agents to the surface. Agents can also be pre-immobilized onto solid supports, such as beads, which then can be added to the receptacles. A response in a cell-type contacted with the beads in the receptacles could subsequently be detected. Mixtures of beads comprising single agents may be combined to form agent mixtures. Alternatively, mixtures of single agents can be immobilized to the beads.

The agents can also be immobilized on or impregnated within a scaffold, which can be placed in the receptacle and then contacted with fluid containing the cells. Suitable scaffolds for use in the embodiments described above, and methods for immobilizing agents thereto or therewithin are described in copending, commonly owned U.S. patent application Ser. No. 10/259,817, filed on Sep. 30, 2002, the entire content of which is incorporated herein by reference.

Receptacles for use in the embodiments described above can take any usual form, but are desirably microwells or tubes. Configurations such as microtitre wells and tubes are particularly useful and allow the simultaneous automated assay of a large number of samples to be performed in an efficient and convenient way. Microtitre wells are capable of extensive automation because of automatic pipetters and plate readers. Other solid phases, particularly other plastic solid supports, may also be used.

In one example, the receptacles comprise the wells of a 96-well microtitre plate (i.e., microwell array). Automatic pipetting equipment for reagent addition and washing steps, and color readers already exist for such microtitre plates as known to those skilled in the art. An example of such an automated device includes a pipetting station and a detection apparatus (e.g., plate reader), wherein the pipetting station is capable of performing sequential operations of adding and removing reagents to the wells at specific time points in a thermostatic environment (i.e., temperature controlled environment).

As described above, agents for use in the embodiments include growth effector molecules that bind receptors on the cell surface or are taken up through ion channels or transports and regulate the growth, replication or differentiation of target cells or tissue. In one example, these agents are cell adhesion ligands and/or extrinsic factors. In still other examples, the agents can be extracellular matrix proteins, extracellular matrix protein fragments, peptides, growth factors, cytokines, and combinations thereof, including an example described in greater detail below including sets and subsets of two and three peptones that are use to optimize cell culture conditions.

Preferred agents are growth factors, extracellular matrix molecules, cytokines, peptides, hormones, metals, chelators or enzymes. Examples of growth factors include, but are not limited to, vascular endothelial-derived growth factor (VEGF), epidermal growth factor (EGF), platelet-derived growth factor (PDGF), transforming growth factors (TGFa, TGFβ), hepatocyte growth factor, heparin binding factor, insulin-like growth factor I or II, fibroblast growth factor, erythropoietin nerve growth factor, bone morphogenic proteins, muscle morphogenic proteins, and other factors known to those skilled in the art. Other suitable growth factors are described in a publication by M. B. Sporn and A. B. Roberts, Eds., entitled “Peptide Growth Factors and Their Receptors I”, published by Springer-Verlag, NY, 1990, the entire content of which is incorporated herein by reference.

Such growth factors can be isolated from tissues using methods well known to those skilled in the art. For example, growth factors can be isolated from tissue or can be produced by recombinant means. Epidermal growth factor can be isolated from the submaxillary glands of mice and Genentech, of San Francisco, Calif., produces TGF-β recombinantly. Other growth factors in both natural and recombinant forms are also available from vendors such as Sigma Chemical Co., of St. Louis, Mo., R&D Systems, of Minneapolis, Minn., BD Biosciences, of San Jose, Calif., and Invitrogen Corporation, of Carlsbad, Calif.

Examples of suitable extracellular matrix molecules for use in the embodiment include vitronectin, tenascin, thrombospondin, fibronectin, laminin, collagens, and proteoglycans. Other extracellular matrix molecules are described in a publication by Kleinman et al., entitled “Use of Extracellular Matrix Components for Cell Culture,” published by Analytical Biochemistry 166:1-13 (1987).

Additional agents useful in the method described above include cytokines, such as the interleukins and GM-colony stimulating factor, and hormones, such as insulin. These are described in the literature referenced above and are commercially available.

Cells for use with the embodiments can be any cells that can potentially respond to the agents or that need the agents for growth. For example, cells can be obtained from established cells lines or separated from isolated tissue. Suitable cells include most epithelial and endothelial cell types, for example, parenchymal cells such as hepatocytes, pancreatic islet cells, fibroblasts, chondrocytes, osteoblasts, exocrine cells, cells of intestinal origin, bile duct cells, parathyroid cells, thyroid cells, cells of the adrenal-hypothalamic-pituitary access, heart muscle cells, kidney epithelial cells, kidney tubular cells, kidney basement membrane cells, nerve cells, blood vessel cells, cells forming bone and cartilage, and smooth and skeletal muscles.

Other useful cells can include stem cells which may undergo a change in phenotype in response to a select mixture of agents. Further suitable cells include blood cells, umbilical cord blood-derived cells, umbilical cord blood-derived stem cells, umbilical cord blood-derived progenitor cells, umbilical cord-derived cells, placenta-derived cells, bone marrow derived cells, and cells from amniotic fluid. The cells can be genetically engineered, and/or cultured with agents in a receptacle, such as the well of a 96-well microtitre plate. These cells can be cultured using any of the numerous cell culture techniques well known to those skilled in the art, such as those described in the text by Freshney, entitled “Cell Culture, A Manual of Basic Technique”, 3^{rd} Edition, published by Wiley-Liss, NY, 1994. Other cell culture media and techniques are well known to those skilled in the art and can also be used in the embodiments of the present invention described above.

The cells can be cultured in the presence of agents which are in a solution or which are bound to a standard tissue culture vessel, such as a microtitre plate. The cells can also be cultured in suspension using agents that have been tethered to beads or fibers, preferably on the order of 10 microns in diameter. These particles, when added to culture medium, would attach to the cells, thereby stimulating their growth and providing attachment signals.

In a specific application, the system and method described above can be applied to identify the best subset of two or three peptones that optimizes cell culture conditions based on a variety of responses. Starting from a list of several possible peptones, an optimization strategy can be developed to identify the best subset of two or three peptones that optimizes cell culture conditions based on a variety of responses such as antibody secretion, cell number, and time to peak antibody secretion. Optimization techniques are further discussed in a publication by Taylor et al., entitled “Automated Assay Optimization With Integrated Statistics And Smart Robotics”, published by Journal Of Bimolecular Screening, 5(4): 213-225, August 2000, in a publication by Wolcke et al., entitled “Miniaturized HTS Technologies—uHTS”, published by Drug Discovery Today, 6 (12): 637-646, Jun. 15, 2001, and in a publication by Lutz et al., entitled “Experimental Design For High-Throughput Screening”, published by Drug Delivery Today, 1 (7): 277-286, July 1996, the entire content of each is incorporated herein by reference.

The system and method can be provided as an automated media optimization technology that enables users to optimize media components (i.e. factors) using a MPM/CATSBA software and robotic liquid-handling platforms. Using such specific factors, the software can automatically create statistically designed experiments in a multi-well plate format. The software then generates the necessary files to prepare the correct experimental conditions using a robotic-liquid-handling platform (e.g. the Biomek FX, Biomek 2000, Tecan Genesis, or any similar platforms). The software and the database it resides on can then be used to automatically categorize and analyze numerous formats of data (i.e. fluorescence, absorbance, cell counts, and so forth). The software user can then perform all relevant statistical analyses in an automated fashion and all relevant reports are automatically generated and stored within the database. After all relevant statistical analyses are performed, the user has the ability to combine results from multiple experiments for a meta-analysis and data mining.

In this example, the system and method initiates an assay development and determination of basic cell culture conditions in a first step. Specifically, the user specifies the factors (i.e. peptones) and their concentrations into the software (i.e. MPM/CATSBA) via a GUI, as noted in block 100 of FIG. 1A. The user selects an appropriate statistical design as noted in block 107, and the software automatically creates the correct experimental protocol including the specific factors and their concentrations as noted in block 108. The user then selects an option in the software that automatically creates necessary computer files that can be imported into by robotic sample preparation platform (e.g., the Biomek FX, Biomek 2000, Tecan Genesis, or any similar platforms) as noted in block 112.

In a second step, a dilution series is provided in 96 well plates to determine optimal concentrations of peptones one-at-a-time. The computer files are used to automatically prepare the correct experimental conditions on the 96 well plates as noted in block 114 of FIG. 1B. Once the agents have been placed into the wells correctly, the robotic system at block 116 dispenses fluid including whole cells into the wells of the microwell array. Assay results are imported into the software and automatically linked to the description of the experiments so that the data is fully annotated and ready for statistical analysis.

Specifically, at block 118 experimental data is acquired which would be indicative of a change in the phenotype of a cell, and is stored in a database at block 120 so that the experimental data is linked to the computer representation of the design. Then at block 122, a processor is utilized which includes an algorithm to compare the stored experimental data to the stored statistical design to identify the optimal concentrations that elicits the desired biological response (i.e., elicited a phenotypic change in the cells). The results of the algorithm comparisons can be stored in a database and displayed to a user at block 124, and can be periodically updated.

The resulting experiments in the 96 well plates are used to identify the best subsets of two and/or three peptones with a verification in shake flasks. That is, the optimization experiments in the 96 well plates determine the best concentrations of the peptones in the best subsets with verification in flasks.

A bake-off then provides the best subsets/best concentrations against customer media and appropriate controls in the 96 well plates followed by verification in shake flasks. Once again returning to FIGS. 1A and 1B, the databases including the customer media and appropriate controls used can be a single integrated or federated database. At block 126, the steps of the process can be repeated with a subset of the best mixtures or a subset of the best agents. Moreover, if desired, the steps can be repeated with a combined subset of best agents and a subset of agents from the best mixtures. Furthermore, at block 128 the steps of the process can be repeated, varying the concentration and/or amounts of the agents in the best mixtures, and scale up the best conditions with additional validation and quality control (QC). Any information acquired from the algorithm comparisons at block 122 can optimally be used to create or revise a biological model at block 130.

The user can perform all relevant statistical analyses in an automated way from information provided by the software application. Reports can then be generated and the results stored in the database. Based on the results of the statistical analysis, the user may return to the first step to start the next experiment or may proceed to scaling up the best media formulation.

In this specific example, the agents, or factors, are limited to peptones, but the system and method described above is general to applications including any reagents or factors that could be added to cell culture media. In the automated optimization application of the embodiment described above, the resulting tables described in greater detail below use an eight peptone set, but the number may be varied without affecting the strategy although specific design parameters would require modifications.

This specific example is directed at the detection of the best subsets of two and three peptones, but the number of peptones to be included in the best subsets evaluation could be increased or decreased within the same strategy although as noted above, specific designs parameters would require modifications. Additionally, the examples below incorporates 96 well plates, however the actual format of the plates could be changed and the overall strategy would still be valid. If larger or smaller plates were used, the designs would need to be revised accordingly.

In this example, the system and method is directed towards peptone combinations of two and three peptones at a time in order to determine which subsets are best. With eight peptones, wherein each is provided having at least two different concentration levels, the embodiment can do all of the two-way combinations (i.e. 8 choose 2=28) at both a higher and lower (i.e. higher/higher and lower/lower) concentration on one plate as shown in TABLE 1, with several wells left over for controls. Additionally, outer wells are not used due to evaporation. For example, in TABLE 1, CG Soy is evaluated in concentrations of 3.0 mg/mL (i.e. lower concentration for CG Soy) and 4.0 mg/mL (i.e. higher concentration for CG Soy) with the remaining peptones, such as Phytone in concentrations of 9.0 mg/mL (i.e. lower concentration for Phytone) and 10.0 mg/mL (i.e. higher concentration for Phytone), respectively.

On the second plate, the system and method is directed towards three-way combinations (i.e. 8 choose 3=56) at a single respective concentration level for each peptone on one plate as shown in TABLE 2, with several wells provided for controls. The controls on each plate allow comparisons between the plates and allow for additional statistical analysis. For example, in TABLE 2, CG Soy is evaluated in a concentration of 3.0 mg/mL (i.e. single concentration for CG Soy) with the remaining peptones, such as Phytone in a concentration of 9.0 mg/mL (i.e. single concentration for Phytone) and Phytone UF in a concentration of 2.0 mg/mL (i.e. single concentration for Phytone UF).

An example array of plate 1 is shown in TABLE 1, wherein the best subsets of two peptones are detected from a set of eight peptones, in addition to a number of control wells and replicated wells from plate 2.

TABLE 1

| | | | | | Phytone | | | | Select | | | | | | Yeastolate | |
|---|---|---|---|---|---|---|---|---|---|---|---|---|---|---|---|---|
| Well ID | CG Soy | | Phytone | | UF | | Proteose 3 | | Soytone | | Wheat | | Yeastolate | | Plus | |
| B02 | 3.0 | mg/mL | 9.0 | mg/mL | 0 | | 0 | | 0 | | 0 | | 0 | | 0 | |
| B03 | 3.0 | mg/mL | 0 | | 2.0 | mg/mL | 0 | | 0 | | 0 | | 0 | | 0 | |
| B04 | 3.0 | mg/mL | 0 | | 0 | | 2.0 | mg/mL | 0 | | 0 | | 0 | | 0 | |
| B05 | 3.0 | mg/mL | 0 | | 0 | | 0 | | 2.0 | mg/mL | 0 | | 0 | | 0 | |
| B06 | 3.0 | mg/mL | 0 | | 0 | | 0 | | 0 | | 7.0 | mg/mL | 0 | | 0 | |
| B07 | 3.0 | mg/mL | 0 | | 0 | | 0 | | 0 | | 0 | | 3.0 | mg/mL | 0 | |
| B08 | 3.0 | mg/mL | 0 | | 0 | | 0 | | 0 | | 0 | | 0 | | 3.0 | mg/mL |
| B09 | 0 | | 0 | | 0 | | 0 | | 0 | | 0 | | 4.0 | mg/mL | 4.0 | mg/mL |
| B10 | 0 | | 0 | | 0 | | 0 | | 0 | | 8.0 | mg/mL | 0 | | 4.0 | mg/mL |
| B11 | 0 | | 0 | | 0 | | 0 | | 0 | | 8.0 | mg/mL | 4.0 | mg/mL | 0 | |
| C02 | 0 | | 9.0 | mg/mL | 2.0 | mg/mL | 0 | | 0 | | 0 | | 0 | | 0 | |
| C03 | 0 | | 9.0 | mg/mL | 0 | | 2.0 | mg/mL | 0 | | 0 | | 0 | | 0 | |
| C04 | 0 | | 9.0 | mg/mL | 0 | | 0 | | 2.0 | mg/mL | 0 | | 0 | | 0 | |
| C05 | 0 | | 9.0 | mg/mL | 0 | | 0 | | 0 | | 7.0 | mg/mL | 0 | | 0 | |
| C06 | 0 | | 9.0 | mg/mL | 0 | | 0 | | 0 | | 0 | | 3.0 | mg/mL | 0 | |
| C07 | 0 | | 9.0 | mg/mL | 0 | | 0 | | 0 | | 0 | | 0 | | 3.0 | mg/mL |
| C08 | 0 | | 0 | | 0 | | 0 | | 0 | | 0 | | 0 | | 0 | |
| C09 | 0 | | 0 | | 0 | | 0 | | 3.0 | mg/mL | 0 | | 0 | | 4.0 | mg/mL |
| C10 | 0 | | 0 | | 0 | | 0 | | 3.0 | mg/mL | 0 | | 4.0 | mg/mL | 0 | |
| C11 | 0 | | 0 | | 0 | | 0 | | 3.0 | mg/mL | 8.0 | mg/mL | 0 | | 0 | |
| D02 | 0 | | 0 | | 2.0 | mg/mL | 2.0 | mg/mL | 0 | | 0 | | 0 | | 0 | |
| D03 | 0 | | 0 | | 2.0 | mg/mL | 0 | | 2.0 | mg/mL | 0 | | 0 | | 0 | |
| D04 | 0 | | 0 | | 2.0 | mg/mL | 0 | | 0 | | 7.0 | mg/mL | 0 | | 0 | |
| D05 | 0 | | 0 | | 2.0 | mg/mL | 0 | | 0 | | 0 | | 3.0 | mg/mL | 0 | |
| D06 | 0 | | 0 | | 2.0 | mg/mL | 0 | | 0 | | 0 | | 0 | | 3.0 | mg/mL |
| D07 | 4.0 | mg/mL | 10.0 | mg/mL | 3.0 | mg/mL | 0 | | 0 | | 0 | | 0 | | 0 | |
| D08 | 0 | | 0 | | 0 | | 3.0 | mg/mL | 0 | | 0 | | 0 | | 4.0 | mg/mL |
| D09 | 0 | | 0 | | 0 | | 3.0 | mg/mL | 0 | | 0 | | 4.0 | mg/mL | 0 | |
| D10 | 0 | | 0 | | 0 | | 3.0 | mg/mL | 0 | | 8.0 | mg/mL | 0 | | 0 | |
| D11 | 0 | | 0 | | 0 | | 3.0 | mg/mL | 3.0 | mg/mL | 0 | | 0 | | 0 | |
| E02 | 0 | | 0 | | 0 | | 2.0 | mg/mL | 2.0 | mg/mL | 0 | | 0 | | 0 | |
| E03 | 0 | | 0 | | 0 | | 2.0 | mg/mL | 0 | | 7.0 | mg/mL | 0 | | 0 | |
| E04 | 0 | | 0 | | 0 | | 2.0 | mg/mL | 0 | | 0 | | 3.0 | mg/mL | 0 | |
| E05 | 0 | | 0 | | 0 | | 2.0 | mg/mL | 0 | | 0 | | 0 | | 3.0 | mg/mL |
| E06 | 0 | | 0 | | 0 | | 3.0 | mg/mL | 3.0 | mg/mL | 8.0 | mg/mL | 0 | | 0 | |
| E07 | 0 | | 0 | | 3.0 | mg/mL | 0 | | 0 | | 0 | | 0 | | 4.0 | mg/mL |
| E08 | 0 | | 0 | | 3.0 | mg/mL | 0 | | 0 | | 0 | | 4.0 | mg/mL | 0 | |
| E09 | 0 | | 0 | | 3.0 | mg/mL | 0 | | 0 | | 8.0 | mg/mL | 0 | | 0 | |
| E10 | 0 | | 0 | | 3.0 | mg/mL | 0 | | 3.0 | mg/mL | 0 | | 0 | | 0 | |
| E11 | 0 | | 0 | | 3.0 | mg/mL | 3.0 | mg/mL | 0 | | 0 | | 0 | | 0 | |
| F02 | 0 | | 0 | | 0 | | 0 | | 2.0 | mg/mL | 7.0 | mg/mL | 0 | | 0 | |
| F03 | 0 | | 0 | | 0 | | 0 | | 2.0 | mg/mL | 0 | | 3.0 | mg/mL | 0 | |
| F04 | 0 | | 0 | | 0 | | 0 | | 2.0 | mg/mL | 0 | | 0 | | 3.0 | mg/mL |
| F05 | 4.0 | mg/mL | 0 | | 0 | | 0 | | 0 | | 0 | | 4.0 | mg/mL | 4.0 | mg/mL |
| F06 | 0 | | 10.0 | mg/mL | 0 | | 0 | | 0 | | 0 | | 0 | | 4.0 | mg/mL |
| F07 | 0 | | 10.0 | mg/mL | 0 | | 0 | | 0 | | 0 | | 4.0 | mg/mL | 0 | |
| F08 | 0 | | 10.0 | mg/mL | 0 | | 0 | | 0 | | 8.0 | mg/mL | 0 | | 0 | |
| F09 | 0 | | 10.0 | mg/mL | 0 | | 0 | | 3.0 | mg/mL | 0 | | 0 | | 0 | |
| F10 | 0 | | 10.0 | mg/mL | 0 | | 3.0 | mg/mL | 0 | | 0 | | 0 | | 0 | |
| F11 | 0 | | 10.0 | mg/mL | 3.0 mg/mL | mg/mL | 0 | | 0 | | 0 | | 0 | | 0 | |
| G02 | 0 | | 0 | | 0 | | 0 | | 0 | | 7.0 | mg/mL | 3.0 | mg/mL | 0 | |
| G03 | 0 | | 0 | | 0 | | 0 | | 0 | | 7.0 | mg/mL | 0 | | 3.0 | mg/mL |
| G04 | 0 | | 0 | | 0 | | 0 | | 0 | | 0 | | 3.0 | mg/mL | 3.0 | mg/mL |
| G05 | 4.0 | mg/mL | 0 | | 0 | | 0 | | 0 | | 0 | | 0 | | 4.0 | mg/mL |
| G06 | 4.0 | mg/mL | 0 | | 0 | | 0 | | 0 | | 0 | | 4.0 | mg/mL | 0 | |
| G07 | 4.0 | mg/mL | 0 | | 0 | | 0 | | 0 | | 8.0 | mg/mL | 0 | | 0 | |
| G08 | 4.0 | mg/mL | 0 | | 0 | | 0 | | 3.0 | mg/mL | 0 | | 0 | | 0 | |
| G09 | 4.0 | mg/mL | 0 | | 0 | | 3.0 | mg/mL | 0 | | 0 | | 0 | | 0 | |
| G10 | 4.0 | mg/mL | 0 | | 3.0 | mg/mL | 0 | | 0 | | 0 | | 0 | | 0 | |
| G11 | 4.0 | mg/mL | 10.0 | mg/mL | 0 | | 0 | | 0 | | 0 | | 0 | | 0 | |

In TABLE 1, eight peptones are used in the evaluation, including CG SOY, Phytone, Phytone UF, Proteose 3, Select Soytone, Wheat, Yeastolate, and Yeastolate Plus. Each peptone is included in either a low concentration or a high concentration. For example, CG Soy is used in concentrations including 3.0 mg/mL as a low concentration, and 4.0 mg/mL as a high concentration. These values can vary between peptones, as shown by comparison with Phytone which is used in concentrations including 9.0 mg/mL as a low concentration, and 10.0 mg/mL as a high concentration. The Well ID number indicates the microwell array well into which the indicated concentrations of peptone combinations are placed.

As illustrated in TABLE 1, a robotic system places the selected combinations of desired peptone concentrations into wells of a microwell array based on the computer representation created in steps 100-112 of FIG. 1A in an automated procedure. As noted above, TABLE 1 represents an application using eight peptones which results in sufficient space for placing all two-way combinations of two peptones (i.e. 8 choose 2=28), having low and high concentrations into the wells of a single plate.

The system and method then acquires experimental data indicative of a phenotypic change in the contacted cells. Specifically, in this example, indicative data includes growth (i.e. proliferation) and secretion of antibodies (i.e. IgG Secretion/Cell proliferation). The system can than can store the experimental data in a database with links to the computer representation of the experimental design. In doing so, an algorithm can then be applied to compare experimental data to statistical designs to identify the best peptone combination, and more specifically, the one best two peptone combination for inclusion in a subset concentration evaluation as described in greater detail below. The procedure can then be repeated for subsets of three peptones from the set of eight peptones.

An example array of plate 2 is shown in TABLE 2, wherein the best subsets of three peptones are detected in addition to a number of control wells and replicated wells from plate 1.

TABLE 2

| | | | | | Phytone | | | | Select | | | | | | Yeastolate | |
|---|---|---|---|---|---|---|---|---|---|---|---|---|---|---|---|---|
| Well ID | CG Soy | | Phytone | | UF | | Proteose 3 | | Soytone | | Wheat | | Yeastolate | | Plus | |
| B02 | 3.0 | mg/mL | 9.0 | mg/mL | 2.0 | mg/mL | 0 | | 0 | | 0 | | 0 | | 0 | |
| B03 | 3.0 | mg/mL | 9.0 | mg/mL | 0 | | 2.0 | mg/mL | 0 | | 0 | | 0 | | 0 | |
| B04 | 3.0 | mg/mL | 9.0 | mg/mL | 0 | | 0 | | 2.0 | mg/mL | 0 | | 0 | | 0 | |
| B05 | 3.0 | mg/mL | 9.0 | mg/mL | 0 | | 0 | | 0 | | 7.0 | mg/mL | 0 | | 0 | |
| B06 | 3.0 | mg/mL | 9.0 | mg/mL | 0 | | 0 | | 0 | | 0 | | 3.0 | mg/mL | 0 | |
| B07 | 3.0 | mg/mL | 9.0 | mg/mL | 0 | | 0 | | 0 | | 0 | | 0 | | 3.0 | mg/mL |
| B08 | 0 | | 0 | | 0 | | 2.0 | mg/mL | 2.0 | mg/mL | 7.0 | mg/mL | 0 | | 0 | |
| B09 | 0 | | 0 | | 0 | | 2.0 | mg/mL | 2.0 | mg/mL | 0 | | 3.0 | mg/mL | 0 | |
| B10 | 0 | | 0 | | 0 | | 2.0 | mg/mL | 2.0 | mg/mL | 0 | | 0 | | 3.0 | mg/mL |
| B11 | 0 | | 0 | | 0 | | 0 | | 0 | | 7.0 | mg/mL | 3.0 | mg/mL | 0 | |
| C02 | 3.0 | mg/mL | 0 | | 2.0 | mg/mL | 2.0 | mg/mL | 0 | | 0 | | 0 | | 0 | |
| C03 | 3.0 | mg/mL | 0 | | 2.0 | mg/mL | 0 | | 2.0 | mg/mL | 0 | | 0 | | 0 | |
| C04 | 3.0 | mg/mL | 0 | | 2.0 | mg/mL | 0 | | 0 | | 7.0 | mg/mL | 0 | | 0 | |
| C05 | 3.0 | mg/mL | 0 | | 2.0 | mg/mL | 0 | | 0 | | 0 | | 3.0 | mg/mL | 0 | |
| C06 | 3.0 | mg/mL | 0 | | 2.0 | mg/mL | 0 | | 0 | | 0 | | 0 | | 3.0 | mg/mL |
| C07 | 0 | | 0 | | 0 | | 2.0 | mg/mL | 0 | | 7.0 | mg/mL | 3.0 | mg/mL | 0 | |
| C08 | 0 | | 0 | | 0 | | 2.0 | mg/mL | 0 | | 7.0 | mg/mL | 0 | | 3.0 | mg/mL |
| C09 | 0 | | 0 | | 0 | | 0 | | 2.0 | mg/mL | 7.0 | mg/mL | 3.0 | mg/mL | 0 | |
| C10 | 0 | | 0 | | 0 | | 0 | | 2.0 | mg/mL | 7.0 | mg/mL | 0 | | 3.0 | mg/mL |
| C11 | 0 | | 9.0 | mg/mL | 0 | | 0 | | 0 | | 0 | | 3.0 | mg/mL | 3.0 | mg/mL |
| D02 | 3.0 | mg/mL | 0 | | 0 | | 2.0 | mg/mL | 2.0 | mg/mL | 0 | | 0 | | 0 | |
| D03 | 3.0 | mg/mL | 0 | | 0 | | 2.0 | mg/mL | 0 | | 7.0 | mg/mL | 0 | | 0 | |
| D04 | 3.0 | mg/mL | 0 | | 0 | | 2.0 | mg/mL | 0 | | 0 | | 3.0 | mg/mL | 0 | |
| D05 | 3.0 | mg/mL | 0 | | 0 | | 2.0 | mg/mL | 0 | | 0 | | 0 | | 3.0 | mg/mL |
| D06 | 0 | | 0 | | 2.0 | mg/mL | 0 | | 0 | | 0 | | 3.0 | mg/mL | 3.0 | mg/mL |
| D07 | 0 | | 0 | | 0 | | 2.0 | mg/mL | 0 | | 0 | | 3.0 | mg/mL | 3.0 | mg/mL |
| D08 | 0 | | 0 | | 0 | | 0 | | 2.0 | mg/mL | 0 | | 3.0 | mg/mL | 3.0 | mg/mL |
| D09 | 0 | | 0 | | 0 | | 0 | | 0 | | 7.0 | mg/mL | 3.0 | mg/mL | 3.0 | mg/mL |
| D10 | 0 | | 9.0 | mg/mL | 0 | | 0 | | 0 | | 7.0 | mg/mL | 0 | | 3.0 | mg/mL |
| D11 | 0 | | 9.0 | mg/mL | 0 | | 0 | | 0 | | 7.0 | mg/mL | 3.0 | mg/mL | 0 | |
| E02 | 3.0 | mg/mL | 0 | | 0 | | 0 | | 2.0 | mg/mL | 7.0 | mg/mL | 0 | | 0 | |
| E03 | 3.0 | mg/mL | 0 | | 0 | | 0 | | 2.0 | mg/mL | 0 | | 3.0 | mg/mL | 0 | |
| E04 | 3.0 | mg/mL | 0 | | 0 | | 0 | | 2.0 | mg/mL | 0 | | 0 | | 3.0 | mg/mL |
| E05 | 0 | | 0 | | 2.0 | mg/mL | 0 | | 0 | | 7.0 | mg/mL | 3.0 | mg/mL | 0 | |
| E06 | 0 | | 0 | | 2.0 | mg/mL | 0 | | 0 | | 7.0 | mg/mL | 0 | | 3.0 | mg/mL |
| E07 | 0 | | 0 | | 0 | | 0 | | 0 | | 0 | | 0 | | 0 | |
| E08 | 0 | | 0 | | 2.0 | mg/mL | 0 | | 2.0 | mg/mL | 0 | | 0 | | 0 | |
| E09 | 0 | | 9.0 | mg/mL | 0 | | 0 | | 2.0 | mg/mL | 0 | | 0 | | 3.0 | mg/mL |
| E10 | 0 | | 9.0 | mg/mL | 0 | | 0 | | 2.0 | mg/mL | 0 | | 3.0 | mg/mL | 0 | |
| E11 | 0 | | 9.0 | mg/mL | 0 | | 0 | | 2.0 | mg/mL | 7.0 | mg/mL | 0 | | 0 | |
| F02 | 3.0 | mg/mL | 0 | | 0 | | 0 | | 0 | | 7.0 | mg/mL | 3.0 | mg/mL | 0 | |
| F03 | 3.0 | mg/mL | 0 | | 0 | | 0 | | 0 | | 7.0 | mg/mL | 0 | | 3.0 | mg/mL |
| F04 | 0 | | 0 | | 2.0 | mg/mL | 0 | | 2.0 | mg/mL | 7.0 | mg/mL | 0 | | 0 | |
| F05 | 0 | | 0 | | 2.0 | mg/mL | 0 | | 2.0 | mg/mL | 0 | | 3.0 | mg/mL | 0 | |
| F06 | 0 | | 0 | | 2.0 | mg/mL | 0 | | 2.0 | mg/mL | 0 | | 0 | | 3.0 | mg/mL |
| F07 | 3.0 | mg/mL | 9.0 | mg/mL | 0 | | 0 | | 0 | | 0 | | 0 | | 0 | |
| F08 | 0 | | 9.0 | mg/mL | 0 | | 2.0 | mg/mL | 0 | | 0 | | 0 | | 3.0 | mg/mL |
| F09 | 0 | | 9.0 | mg/mL | 0 | | 2.0 | mg/mL | 0 | | 0 | | 3.0 | mg/mL | 0 | |
| F10 | 0 | | 9.0 | mg/mL | 0 | | 2.0 | mg/mL | 0 | | 7.0 | mg/mL | 0 | | 0 | |
| F11 | 0 | | 9.0 | mg/mL | 0 | | 2.0 | mg/mL | 2.0 | mg/mL | 0 | | 0 | | 0 | |
| G02 | 3.0 | mg/mL | 0 | | 0 | | 0 | | 0 | | 0 | | 3.0 | mg/mL | 3.0 | mg/mL |
| G03 | 0 | | 0 | | 2.0 | mg/mL | 2.0 | mg/mL | 2.0 | mg/mL | 0 | | 0 | | 0 | |
| G04 | 0 | | 0 | | 2.0 | mg/mL | 2.0 | mg/mL | 0 | | 7.0 | mg/mL | 0 | | 0 | |
| G05 | 0 | | 0 | | 2.0 | mg/mL | 2.0 | mg/mL | 0 | | 0 | | 3.0 | mg/mL | 0 | |
| G06 | 0 | | 0 | | 2.0 | mg/mL | 2.0 | mg/mL | 0 | | 0 | | 0 | | 3.0 | mg/mL |
| G07 | 0 | | 9.0 | mg/mL | 2.0 | mg/mL | 0 | | 0 | | 0 | | 0 | | 3.0 | mg/mL |
| G08 | 0 | | 9.0 | mg/mL | 2.0 | mg/mL | 0 | | 0 | | 0 | | 3.0 | mg/mL | 0 | |
| G09 | 0 | | 9.0 | mg/mL | 2.0 | mg/mL | 0 | | 0 | | 7.0 | mg/mL | 0 | | 0 | |
| G10 | 0 | | 9.0 | mg/mL | 2.0 | mg/mL | 0 | | 2.0 | mg/mL | 0 | | 0 | | 0 | |
| G11 | 0 | | 9.0 | mg/mL | 2.0 | mg/mL | 2.0 | mg/mL | 0 | | 0 | | 0 | | 0 | |

In TABLE 2, the eight peptones of TABLE 1 are used again in the evaluation, including CG SOY, Phytone, Phytone UF, Proteose 3, Select Soytone, Wheat, Yeastolate, and Yeastolate Plus. In this evaluation, each peptone is included in a single concentration for each respective peptone. For example, CG Soy is used in a single concentration value of 3.0 mg/mL. As noted above, these values can vary between peptones, as shown by comparison with Phytone which is used in a single concentration value of 9.0 mg/mL. Also as noted above, the Well ID number indicates the microwell array well into which the indicated concentrations of peptone combinations are placed.

As illustrated in TABLE 2, a robotic system once again places the selected combinations of desired peptone concentrations into wells of a microwell array based on the computer representation created in steps 100-112 of FIG. 1A in an automated procedure. As noted above, TABLE 2 represents an application using eight peptones which results in sufficient space for placing all three-way combinations of three peptones (i.e. 8 choose 3=56), having single respective concentrations into the wells of a single plate.

As with TABLE 1, the system and method then acquires experimental data indicative of a phenotypic change in the contacted cells, and stores the experimental data in a database with links to the computer representation of the experimental design. In doing so, an algorithm can then be applied to compare experimental data to statistical designs to identify the best peptone combinations, and more specifically, the three best three peptone combinations for inclusion in a subset concentration evaluation as described in greater detail below.

In this example, the system and method is applied to determine optimum concentrations of subset combinations of two and three peptones selected from the group of eight (i.e. optimization). The result of the best subset experiments above provides several combinations of two and three peptones together that are determined as being the best out of those tested. These subsets of peptones will be carried on to another automated experiment as described in greater detail below, in which the best concentrations will be determined for each subset using standard statistical methods as noted in block 126 and/or 128 of FIG. 1B.

The following protocol of TABLE 3 is a generic template for an optimization plate examining one best subset of two peptones as resulting from TABLE 1, and three best subsets of three peptones as resulting from TABLE 2. The peptones in the different sets may overlap. For each set of peptones, a central composite design is laid out on the plate. Using the inner 60 wells of the plate, various combinations of two and three peptone best subsets can be evaluated for an optimum concentration.

TABLE 3

| Well ID | F01 | F02 | F03 | F04 | F05 | F06 | F07 | F08 | F09 | F10 | F11 | Design ID | Sample typ |
|---|---|---|---|---|---|---|---|---|---|---|---|---|---|
| B02 | 0 | 0 | NA | NA | NA | NA | NA | NA | NA | NA | NA | 1 | test |
| B03 | −1 | −1 | NA | NA | NA | NA | NA | NA | NA | NA | NA | 1 | test |
| C02 | −1 | 1 | NA | NA | NA | NA | NA | NA | NA | NA | NA | 1 | test |
| C03 | 1 | −1 | NA | NA | NA | NA | NA | NA | NA | NA | NA | 1 | test |
| D02 | 1 | 1 | NA | NA | NA | NA | NA | NA | NA | NA | NA | 1 | test |
| D03 | −1.41 | 0 | NA | NA | NA | NA | NA | NA | NA | NA | NA | 1 | test |
| E02 | 1.41 | 0 | NA | NA | NA | NA | NA | NA | NA | NA | NA | 1 | test |
| E03 | NA | NA | NA | NA | NA | NA | NA | NA | NA | NA | NA | NA | positive control |
| F02 | 0 | −1.41 | NA | NA | NA | NA | NA | NA | NA | NA | NA | 1 | test |
| F03 | 0 | 1.41 | NA | NA | NA | NA | NA | NA | NA | NA | NA | 1 | test |
| G02 | 0 | 0 | NA | NA | NA | NA | NA | NA | NA | NA | NA | 1 | test |
| B04 | NA | NA | 0 | 0 | 0 | NA | NA | NA | NA | NA | NA | 2 | test |
| B05 | NA | NA | −1 | −1 | −1 | NA | NA | NA | NA | NA | NA | 2 | test |
| B06 | NA | NA | −1 | −1 | 1 | NA | NA | NA | NA | NA | NA | 2 | test |
| B07 | NA | NA | −1 | 1 | −1 | NA | NA | NA | NA | NA | NA | 2 | test |
| B08 | NA | NA | −1 | 1 | 1 | NA | NA | NA | NA | NA | NA | 2 | test |
| B09 | NA | NA | 1 | −1 | −1 | NA | NA | NA | NA | NA | NA | 2 | test |
| B10 | NA | NA | 1 | −1 | 1 | NA | NA | NA | NA | NA | NA | 2 | test |
| B11 | NA | NA | 1 | 1 | −1 | NA | NA | NA | NA | NA | NA | 2 | test |
| C04 | NA | NA | 1 | 1 | 1 | NA | NA | NA | NA | NA | NA | 2 | test |
| C05 | NA | NA | −1.68 | 0 | 0 | NA | NA | NA | NA | NA | NA | 2 | test |
| C06 | NA | NA | 1.68 | 0 | | NA | NA | NA | NA | NA | NA | 2 | test |
| C07 | NA | NA | 0 | −1.68 | 0 | NA | NA | NA | NA | NA | NA | 2 | test |
| C08 | NA | NA | 0 | 1.68 | 0 | NA | NA | NA | NA | NA | NA | 2 | test |
| C09 | NA | NA | 0 | 0 | −1.68 | NA | NA | NA | NA | NA | NA | 2 | test |
| C10 | NA | NA | 0 | 0 | 1.68 | NA | NA | NA | NA | NA | NA | 2 | test |
| C11 | NA | NA | 0 | 0 | 0 | NA | NA | NA | NA | NA | NA | 2 | test |
| D04 | NA | NA | NA | NA | NA | 0 | 0 | 0 | NA | NA | NA | 3 | test |
| D05 | NA | NA | NA | NA | NA | −1 | −1 | −1 | NA | NA | NA | 3 | test |
| D06 | NA | NA | NA | NA | NA | −1 | −1 | 1 | NA | NA | NA | 3 | test |
| D07 | NA | NA | NA | NA | NA | −1 | 1 | −1 | NA | NA | NA | 3 | test |
| D08 | NA | NA | NA | NA | NA | −1 | 1 | 1 | NA | NA | NA | 3 | test |
| D09 | NA | NA | NA | NA | NA | 1 | −1 | −1 | NA | NA | NA | 3 | test |
| D10 | NA | NA | NA | NA | NA | NA | NA | NA | NA | NA | NA | NA | positive control |
| D11 | NA | NA | NA | NA | NA | 1 | −1 | 1 | NA | NA | NA | 3 | test |
| E04 | NA | NA | NA | NA | NA | 1 | 1 | −1 | NA | NA | NA | 3 | test |
| E05 | NA | NA | NA | NA | NA | 1 | 1 | 1 | NA | NA | NA | 3 | test |
| E06 | NA | NA | NA | NA | NA | −1.68 | 0 | 0 | NA | NA | NA | 3 | test |
| E07 | NA | NA | NA | NA | NA | 1.68 | 0 | | NA | NA | NA | 3 | test |
| E08 | NA | NA | NA | NA | NA | 0 | −1.68 | 0 | NA | NA | NA | 3 | test |
| E09 | NA | NA | NA | NA | NA | 0 | 1.68 | 0 | NA | NA | NA | 3 | test |
| E10 | NA | NA | NA | NA | NA | 0 | 0 | −1.68 | NA | NA | NA | 3 | test |
| E11 | NA | NA | NA | NA | NA | 0 | 0 | 1.68 | NA | NA | NA | 3 | test |
| F04 | NA | NA | NA | NA | NA | 0 | 0 | 0 | NA | NA | NA | 3 | test |
| F05 | NA | NA | NA | NA | NA | NA | NA | NA | 0 | 0 | 0 | 4 | test |
| F06 | NA | NA | NA | NA | NA | NA | NA | NA | −1 | −1 | −1 | 4 | test |
| F07 | NA | NA | NA | NA | NA | NA | NA | NA | −1 | −1 | 1 | 4 | test |
| F08 | NA | NA | NA | NA | NA | NA | NA | NA | −1 | 1 | −1 | 4 | test |
| F09 | NA | NA | NA | NA | NA | NA | NA | NA | −1 | 1 | 1 | 4 | test |
| F10 | NA | NA | NA | NA | NA | NA | NA | NA | 1 | −1 | −1 | 4 | test |
| F11 | NA | NA | NA | NA | NA | NA | NA | NA | 1 | −1 | 1 | 4 | test |
| G03 | NA | NA | NA | NA | NA | NA | NA | NA | 1 | 1 | −1 | 4 | test |
| G04 | NA | NA | NA | NA | NA | NA | NA | NA | 1 | 1 | 1 | 4 | test |
| G05 | NA | NA | NA | NA | NA | NA | NA | NA | −1.68 | 0 | 0 | 4 | test |
| G06 | NA | NA | NA | NA | NA | NA | NA | NA | 1.68 | 0 | 0 | 4 | test |
| G07 | NA | NA | NA | NA | NA | NA | NA | NA | 0 | −1.68 | 0 | 4 | test |
| G08 | NA | NA | NA | NA | NA | NA | NA | NA | 0 | 1.68 | 0 | 4 | test |
| G09 | NA | NA | NA | NA | NA | NA | NA | NA | 0 | 0 | −1.68 | 4 | test |
| G10 | NA | NA | NA | NA | NA | NA | NA | NA | 0 | 0 | 1.68 | 4 | test |
| G11 | NA | NA | NA | NA | NA | NA | NA | NA | 0 | 0 | 0 | 4 | test |

In TABLE 3, the values reflect the coded levels of the peptones used. The peptones in TABLE 3 are assigned as generic factors, F01, F02, . . . , F11. Although only eight peptones were included in this example, the peptones in the different subsets may overlap. In this example, F01 and F02 represent the peptones included in the best subset of two peptones to now be optimized as determined from TABLE 1. The subsets F03-F05, F06-F08, and F09-F11 represent the three best subsets of three peptones to now be optimized as determined from TABLE 2.

The Well ID number of TABLE 3 indicates the microwell array well into which the indicated concentrations of peptone combinations are placed. The Design ID column indicates which subset concentrations are being varied. For example, the Design ID column has the value of 1 for all of the wells in which the concentrations of the subset of two peptones are varied. The Design ID column has values 2, 3 and 4 for the wells in which the concentrations of the three subsets of three peptones are varied (i.e. F03-F05, F06-F08, and F09-F11), respectively. Whenever an NA is present in TABLE 3, the indicated factor, or peptone, is not included in that well.

With reference to TABLE 3, the first column indicates the Well ID number for each of the experimental runs in the 96 well plate. There are 60 runs in this example. The numbers in TABLE 3 in the columns labeled F01, F02, . . . , F11 (−1.41, −1, 0, 1, 1.41, etc.) represent coded values for the levels, or concentrations, of the factors, or peptones, respectively. From gathered information, a range of possible optimum concentrations is determined for each peptone, within which an optimum concentration is believed to exist. This range is assigned relative values using techniques such as Response Surface Methodology (RSM) as described in greater detail below.

In this example, for columns F01 and F02, the values of −1.41 and 1.41 correspond to the maximum and minimum concentrations that are hypothesized to span the range of possible optimum concentrations for the first two peptones, respectively. The concentrations of the peptones that correspond to the coded values of −1, 0, and 1 lie between the maximum and minimum concentrations of −1.41 and 1.41, and can be determined by a simple linear transformation. For example, the coded level of zero corresponds to the concentration midway between the maximum and minimum concentrations.

For the columns F03 to F11, the values of −1.68 and 1.68 correspond to the maximum and minimum concentrations that are hypothesized to span the range of possible optimum concentrations for the corresponding peptones assigned as F03 to F11. The concentrations corresponding to −1, 0, and 1 can be determined as described above. A specific example of a procedure for defining such ranges and subsequently assigning relative values is described in greater detail below.

For example, if from TABLE 1, the best combination of two peptones is found to be CG Soy at a concentration of 3.0 mg/mL and Phytone at a concentration of 9.0 mg/mL, these values could then be applied to the optimization of TABLE 3. As noted above for TABLE 3, F01 and F02 represent the peptones included in the best subset of two peptones as determined from TABLE 1 to be optimized.

In the optimization experiment, a concentration range is determined for each peptone, within which an optimum concentration is believed to exist and this range is assigned relative values. When determining this range, the current best values (i.e. from TABLE 1) can be chosen as a center value, and higher and lower values can then be selected to define the range around the current best values to explore for the optimum. The range should be wide enough to include a best estimate as to the true optimum, but narrow enough to provide a good statistical model. In many applications, this may require inputs from skilled users, such as cell biologists and statisticians.

For the above example, the range for CD Soy for use in TABLE 3 can be assigned relative values based upon the following defined range.
X₀=0 [symbol] corresponds to a concentration of 3.0 mg/mL of CG Soy
X₋₁=−1 [symbol] corresponds to a concentration of 2.0 mg/mL of CG Soy
X₊₁=+1 [symbol] corresponds to a concentration of 4.0 mg/mL of CG Soy
   Where X₀ represents the center of the range for CG Soy, X₋₁ represents an increment in the lower range, and X₊₁ represents an increment in the upper range.

A unit change of one in coded values is 1 mg/mL in concentration values, therefore,
X_{-1.41}=−1.41 [symbol] 3.0−(1.41×1.0)=concentration of 1.59 mg/mL CG Soy
X_{+1.41}=+1.41 [symbol] 3.0+(1.41×1.0)=concentration of 4.41 mg/mL CG Soy
   Where X_{-1.41} represents a lower range boundary for CG Soy, and X_{+1.41} represents an upper range boundary. Also, for the Phytone values, the same procedure can be applied.
Y₀=0 [symbol] corresponds to a concentration of 9.0 mg/mL of Phytone
Y₋₁=−1 [symbol] corresponds to a concentration of 7.0 mg/mL of Phytone
Y₊₁=+1 [symbol] corresponds to a concentration of 11 mg/mL of Phytone

A unit change of one in coded values is 2 mg/mL in concentration values, therefore,
Y_{-1.41}=−1.41 [symbol] 9.0−(1.41×2.0)=concentration of 6.18 mg/mL of Phytone
Y_{+1.41}=+1.41 [symbol] 9.0+(1.41×2.0)=concentration of 11.82 mg/mL of Phytone

A similar procedure applies to the best combination of three peptones found in TABLE 2. As noted above for TABLE 3, the subsets F03-F05, F06-F08, and F09-F11 represent the three best subsets of three peptones as determined from TABLE 2 to be optimized.

For example, if from TABLE 2, the best combination of three peptones is found to be CG Soy at a concentration of 3.0 mg/mL, Phytone UF at a concentration of 2.0 mg/mL and Wheat at a concentration of 7.0 mg/mL, these values could then be applied to the optimization of TABLE 3. As above, the range is selected as a best estimate as to a region that should contain the optimum. An example calculation for one of these three peptone ranges is presented below. The range for Wheat for use in TABLE 3 can be assigned relative values based upon the following defined range.

Z₀=0 corresponds to a concentration of 7.0 mg/mL of Wheat
Z₋₁=−1 corresponds to a concentration of 6.5 mg/mL of Wheat
Z₊₁=+1 corresponds to a concentration of 7.5 mg/mL of Wheat

A unit change of one in coded values therefore is 0.5 mg/mL in concentration values, therefore,
Z_{-1.68}=−1.68 [symbol] 7.0−(1.68×0.5)=7.0−0.84=6.16
Z_{+1.68}=+1.68 [symbol] 7.0+(1.68×0.5)=7.0+0.84=7.84

In many applications of the above embodiment, different concentration levels may be chosen for the same factor in the two and three variable optimization experiments. That is, where a factor is present in both a best combination of two and three peptones, and therefore used in multiple places in TABLE 3, the ranges of the single peptone in TABLE 3 need not be identical.

The generic factor names are provided in the top row of TABLE 3 and correspond to various subsets of the peptones listed in TABLES 1 and 2 in this example. In particular, the actual peptones in the best subset of two peptones resulting from the evaluation of the peptones in TABLE 1 would be substituted for the generic factors F01 and F02. The actual peptones in the first best subset of three peptones resulting from the evaluation of the peptones in TABLE 2 would be substituted for the generic factors F03, F04, and F05, and so on. Since the same peptone may appear in multiple best subsets, the same peptone may correspond to more than one of the generic factor names F01, F02, . . . F11.

As with TABLES 1 and 2, a robotic system places the selected subsets of desired peptone concentration variations into wells of a microwell array in an automated procedure. The evaluation of TABLE 3 results in sufficient space for placing two-way combinations of two peptones having minimum, or low (i.e. −1.41), mid-low (i.e. −1), mid (i.e. 0), mid-high (i.e. 1) and maximum, or high (i.e. 1.41) concentration levels, and placing three-way combinations of three peptones having minimum, or low (i.e. −1.68), mid-low(i.e. −1), mid (i.e. 0), mid-high (i.e. 1) and maximum, or high (i.e. 1.68) concentrations into the wells of a single plate. These concentrations however may not necessarily correspond to the concentrations shown in TABLES 1 and 2 for the same peptones, but as noted above, are concentrations that are hypothesized to span the range of possible optimum concentrations for a specific peptone.

The system and method then acquires experimental data indicative of a phenotypic change in the contacted cells and stores the experimental data in a database with links to the computer representation of the experimental design. In doing so, an algorithm can then be applied to compare experimental data to statistical designs to identify the best peptone concentration values.

The experiments can be further repeated with a subset to arrive at an optimum subset of factors for producing a desired response in a cell. Moreover, the experiment can be repeated wherein the concentration of the agents are varied. Follow-up experiments can also be performed with the subset of single agents that had statistically significant main effects or by combining a subset of the best single agents with a subset identified in the best mixtures.

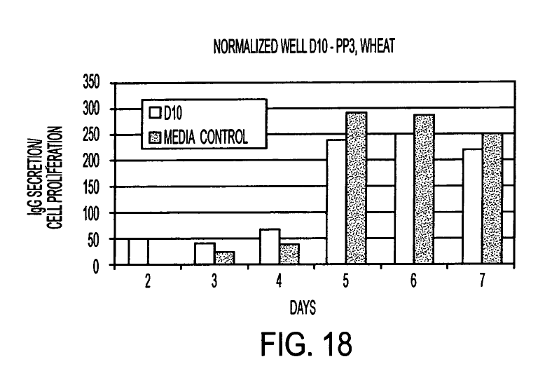

One example of the results provided by the above embodiment are illustrated in FIGS. 18, 19 and 20. The best well analysis of two peptone combinations is shown in FIGS. 18, 19 and 20. Specifically, the data from experimenting with eight media components were analyzed and the best well analysis showed three of the peptones as consistently having a positive effect, Proteose 3, Wheat and Select Soytone.

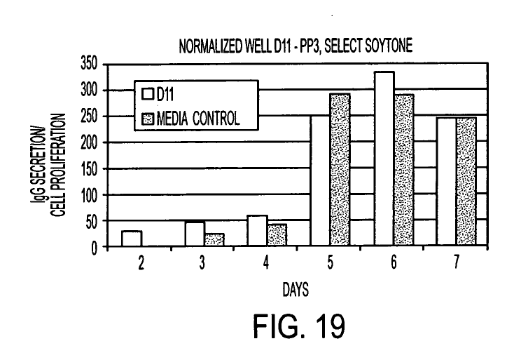

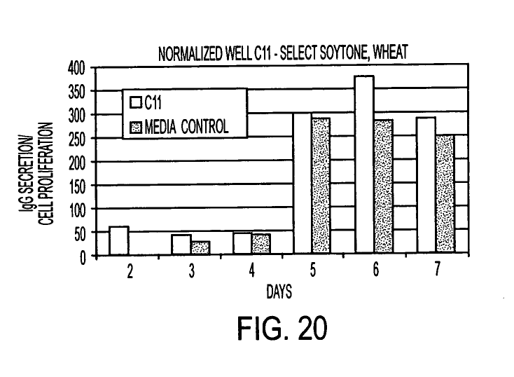

FIG. 18 illustrates results provided by Well ID number D10 of TABLE 1 in which a concentration of 3.0 mg/mL of Proteose 3 was combined with a concentration of 8.0 mg/mL of Wheat. Likewise FIG. 19 illustrates results provided by Well ID number D11 of TABLE 1 in which a concentration of 3.0 mg/mL of Proteose 3 was combined with a concentration of 3.0 mg/mL of Select Soytone. FIG. 20 illustrates results provided by Well ID number C11 of TABLE 1 in which a concentration of 3.0 mg/mL of Select Soytone was combined with a concentration of 8.0 mg/mL of Wheat. The analysis shows these three peptones have a positive effect on HB67 cells and IgG Secretion and Proliferation. Subsets of these three peptones can then be further evaluated using the automated optimization template of TABLE 3 to determine optimum concentrations.

The embodiment described above can be completed in less time than conventional experiments. In particular, all of the best subsets of two peptones can be evaluated on a single plate (i.e. TABLE 1), and all of the best subsets of three peptones can be evaluated on a separate single plate (i.e. TABLE 2). Both of these plates, including replicates thereof, provide results (i.e. subsets) that can be evaluated at the same time in a single experiment on yet another separate plate (i.e. TABLE 3). The automated implementation of this is much faster and more efficient than conventional experiments that are not conducted in multiwell plates. Because the subsets of each size (i.e. two and three) are all evaluated on the same plate, respectively, the data obtained is more directly comparable and reliable. In addition, the follow-up optimization experiment allows the several subsets of two and three peptones to be optimized in the same experiment on the same plate. This is more efficient than conducting the experiments on separate plates, at separate times, and/or in alternative formats to multiwell plates.

Using a software package such as the MPM/CATSBA software, the above embodiments of the present invention remove many of the inherent human errors in cell culture and cellular experimentation through the automated implementation of an optimization strategy. The embodiments allow the implementation of software that makes the physical plate layouts from a complex statistical design in an automated fashion. The complex plate layouts then enable an automated evaluation and determination of solutions to best subset problems in a more efficient manner than currently available. Specifically, in this example the automated implementation of the optimization strategy is used to efficiently identify the best subset of peptone combinations, and thereafter, the best peptone concentrations that optimize cell culture conditions based upon antibody secretion, cell number and time to peak antibody secretion values.

Through a combined knowledge of experimental design and robotics for automated sample preparation, the embodiments integrate computers into traditional cell culture and use this technology to optimize biological systems as opposed to simply optimizing assay conditions.

All plate layouts, liquid-handling commands, and data analysis functions are automatically generated using the software. This automated platform removes most human errors from the experimental process. Prior to using this technology, experiments were either manually programmed into a robotic liquid-handling platform or experiments were created by hand on the benchtop. Both of these experimental approaches are highly likely to contain inherent errors due to manual manipulation and programming errors.

Additionally, the MPM software automatically analyzes all data and may be used to suggest follow-up optimization experiments. This system allows solutions to more complex media optimization problems in a more highly efficient manner. Additionally, the strategy for picking best subsets and jointly optimizing the concentrations for those subsets is novel in both design and implementation. This results in the ability to create complex plate layouts in an automated fashion and leads to very different experiments and observations than would be available in a manual system. For example, synergistic effects can be observed in certain combinations of media components.

The above embodiment further provides much faster pipetting speeds, all providing greater cost savings, improved data analysis times and robotic programming times. The reagent cost savings is calculated by multiplying the number of repeats required by the number of optimization experiments required for each experimental approach, then dividing the conventional result by the above optimization result.

The embodiment described above could be implemented from a customer location that is remote from the actual laboratory where the experiments are being performed. This could involve a web-based interface or the distribution of a thick-client software application to the customer. The level of interaction could range from as simple as dynamically generated reports showing the current status of the optimization to complete customer control of the process. Additionally, the embodiments are applicable to custom media optimization services, as well as custom data, reagent and experimental design management services.

Additional statistically designed experiments in accordance with the embodiments of the present invention are described in greater detail below.

### EXAMPLES

### Example 1

### Coupling of Hyaluronic Acid to an Amine-Rich Tissue Culture Surface

An oxygen/nitrogen plasma is used by Becton Dickinson Labware to create PRIMARIA™ tissue culture products. In particular, oxygen/nitrogen plasma treatment of polystyrene products results in incorporation of oxygen- and nitrogen-containing functional groups, such as amino and amide groups. For this experiment, HA was coupled to the amine-rich surface on PRIMARIA™ multi-well plates through carboxyl groups on HA using carbodiimide bioconjugates chemistries well known in the art, such as those described in “Protein Immobilization: Fundamentals and Applications” Richard S. Taylor, Ed. (M. Dekker, NY, 1991) or as described in copending, commonly owned U.S. application Ser. No. 10/259,797, filed Sep. 30, 2002.

### Example 2

### Coupling of ECM Proteins to Hyaluronic Acid

ECM agents were covalently attached to the HA polymer tethered to the culture surface from Example 1. In particular, aldehyde groups were created on HA by oxidation using the periodate procedure described in E. Junowicz and S. Charm, “The Derivatization of Oxidized Polysaccharides for Protein Immobilization and Affinity Chromotography,” Biochimica et. Biophysica Acta, Vol. 428: 157-165 (1976). This procedure entailed adding sodium periodate to a solution of HA, thus activating the terminal sugar. Subsequently, the activated HA was coupled to the amine groups on the ECM proteins using standard immobilization chemistries, such as those described in “Protein Immobilization: Fundamentals and Applications” Richard F. Taylor, Ed. (M. Dekker, NY, 1991) or copending U.S. application Ser. No. 10/259,797, filed Sep. 30, 2002.

### Example 3

### Use of a Statistically Designed Experiment (Mixture Design) to Screen 10 Different ECM Proteins Simultaneously

In the present example, the statistical design is a mixture design. This design was used to identify pairs of factors, or single factors that had a positive effect on a cell response, and allows us to look at interactions between two ECMs. In this example, 10 single ECMs, each representing a single “factor” are used to created ECM mixtures for placement into the wells of a 96-well plate as shown in FIG. 7. The ECMs covalently attach to biocompatible polymers on the culture surface (see Examples 1 and 2). It is noted that without a statistical design for the experiment, it would take 2¹⁰ (1024) single experiments, or eleven 96-well plates, to test each of the 10 ECMs together with the others against a given cell-type.

In this example, a group of 10 adhesion ligands was selected and a 96-well array was chosen as the format for this screen. To eliminate border effects due to uneven evaporation, only the inner 60 wells of the 96-well array are to be used for the experiment. Wells in the outer rows and columns of the plate can thus be used for suitable controls.

The following 10 adhesion ligands were selected based on their common use as cell culture reagents, commercial availability and price: Collagen I (CI), Collagen III (CIII), Collagen IV (CIV), Collagen VI (CVI), elastin (ELA), fibronectin (FN), vitronectin (VN), laminin (LAM), polylysine (PL), and polyornithine (PO).

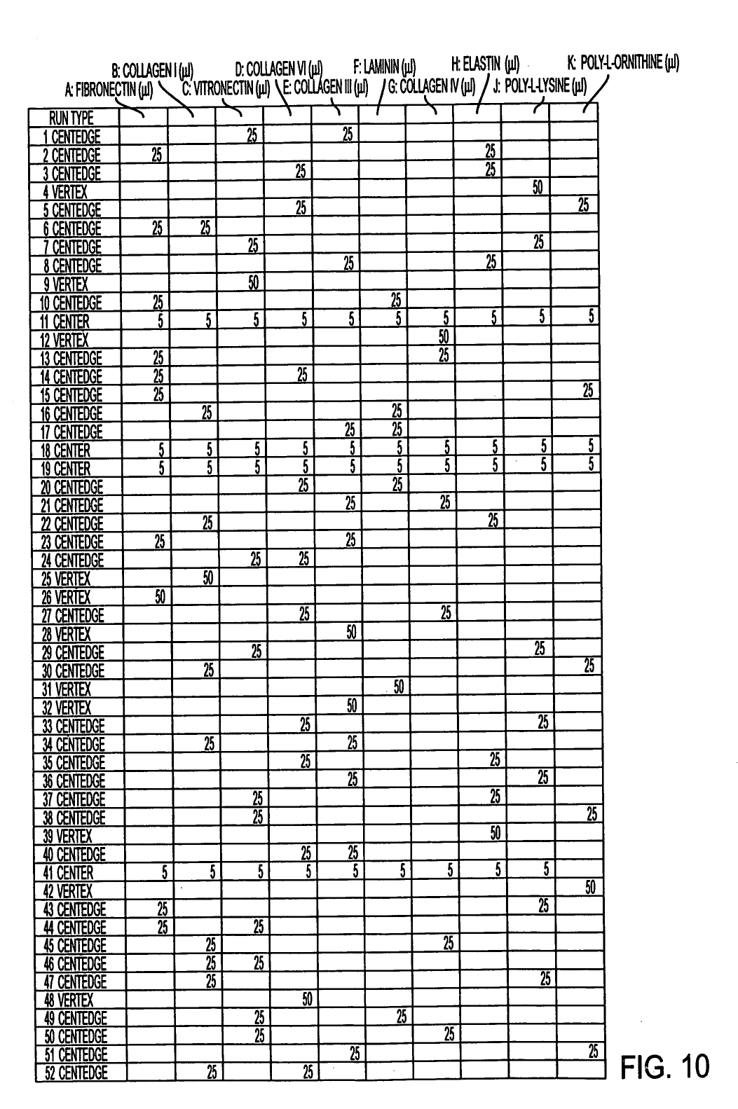

A statistical design was developed with special consideration of the surface chemistry requirements. In particular, in this experiment the scenario shown in FIG. 9 was used, wherein the overall adhesion ligand density was kept constant from well to well and only the adhesion ligand composition was allowed to change. In other words, the concentration of a single adhesion ligand could be different from well to well, but each well has the same amount of adhesion ligand immobilized overall. This scenario is further described above. An example of such design is shown in the spreadsheet in FIG. 10. The spreadsheet serves as a computer representation of the design which is stored in a database. The top row in FIG. 9 lists the 10 factors (A-K) used in this particular screen, and their corresponding identities. In the spreadsheet shown, Factor A represents fibronectin, Factor B represents Collagen I, etc. The first column is a list of the experimental points that translate into a well in the 96-well plate, e.g., 52 wells in this case. The numbers in the spreadsheet are the factor levels. In this example, these levels are the actual volumes (in μL) of factor that are added to a particular well. In this particular design, factors get added to the wells at three volumes, e.g., 5 μL, 25 μL, or 50 μL. The total well volume in this case is 50 μL. Thus, for wells where one factor is added at 50 μL, the final well composition will comprise a single adhesion ligand covalently immobilized on the well surface. Accordingly, if 25 μL of a factor is added to a well, a second factor is added at 25 μL also, and the final well composition will comprise a mixture of two different cell adhesion ligands covalently immobilized on the well surface. When 5 μL of a factor are added, nine other factors are added at 5 μL each, as well, thus resulting in wells that comprise a mixture of all 10 cell adhesion ligands on the well surface. These experimental points containing all 10 adhesion ligands are called “mid points” and are an integral part of the statistical design in this example.

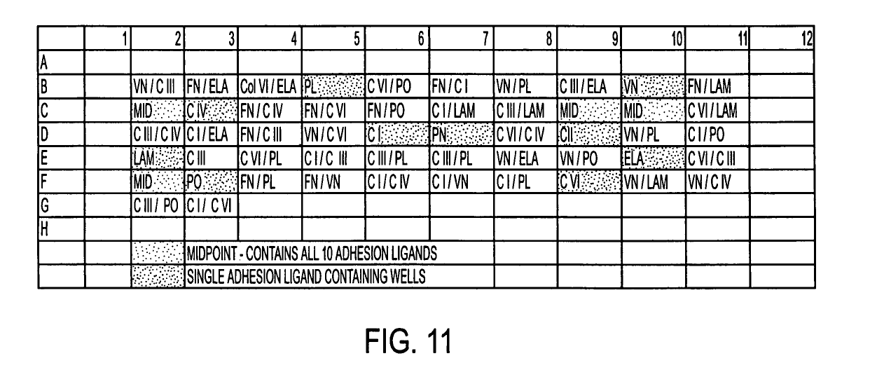

With reference now to FIG. 11, a 96-well plate layout is shown, which was translated from the particular statistical design shown in FIG. 10. In particular, the 96-well plate includes the well compositions indicated in FIG. 10, e.g., cell adhesion ligand combinations immobilized at the bottom of each well. In particular, the experimental runs in FIG. 10 correspond to rows/columns in FIG. 11, as follows: runs 1-10 in the design in FIG. 10 represent row B, columns 2-11, respectively on the array in FIG. 11; runs 11-20 represent row C, columns 2-11; runs 21-30 represent row D, columns 2-11; runs 31-40 represent row E, columns 2-11; runs 41-50 represent row F, columns 2-11; and runs 51 and 52 represent row G, columns 2 and 3, respectively. As shown by the statistical design in FIG. 10 and the corresponding 96-well plate layout in FIG. 11 it is an embodiment of the present invention that, in addition to mixtures of agents, single agents can be placed in the receptacles.

### Example 4

### ECM Screen Specific to MC3T3-E1 Osteoblast Cells

MC3T3-E1 cells, originated from Dr. L. D. Quarles, Duke University, and were kindly provided by Dr. Gale Lester, University of North Carolina at Chapel Hill. These cells were grown using standard cell culture techniques. MC3T3-E1 is a well-characterized and rapidly growing osteoblast cell line that was chosen because it attaches aggressively to most commonly used tissue culture surfaces.

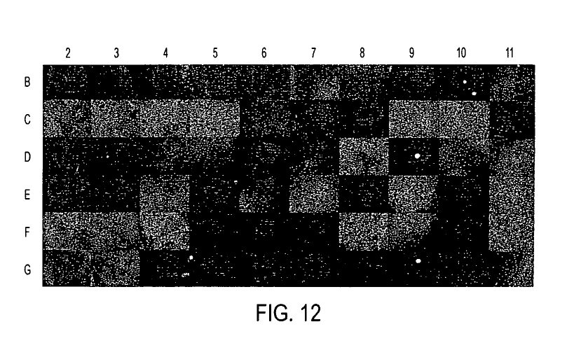

Cells were removed from cell culture flasks using trypsin-EDTA according to methods well known in the art. Cells were enumerated, spun down and resuspended in media containing no serum or, alternatively, in media containing 10% fetal calf serum. Cells were plated into the wells of a 96-well microarray according to the layout shown in FIG. 11 and described in Example 3 above. The seeding density was about 10,000 cells per well. Cells were incubated on the plates overnight at 37° C. The following day, media and any cells not adhering to the immobilized agents on the well surfaces were removed. Any adhered cells were fixed by exposure to formalin for at least 15 minutes. Propidium iodite was used to fluorescently label the nuclei of said fixed adhered cells. A fluorescent microscope (Discovery-1, Universal Imaging Corporation, a subsidiary of Molecular Devices, Downingtown, Pa.) was used to acquire images of the fluorescently labeled cells attached to the wells in the ECM screening plate. An example of an image acquired from a 96-well plate is shown in FIG. 12. In particular, the layout is the same as that shown in FIG. 11, except that row G, column 4-11 are used as control wells. In FIG. 12, MC3T3-E1 cells in 10% fetal calf serum-containing media were placed into wells containing mixtures of agents that had been tethered to a hyaluronic acid surface, with the exception that wells G4-G9 contained a hyaluronic acid surface only and wells G10 and G₁₁ comprised tissue culture grade polystyrene only. As expected, the hyaluronic acid surface only in wells G4-G9 prevented cell adhesion. Cell adhesion to the polystyrene surfaces in wells G10 and G11 was, in this example, surprisingly low. In contrast, some wells containing cell adhesion ligands showed strong cell adhesion, as can be seen by the large number of white spots, each of which represents the nucleus of an adhered cell.

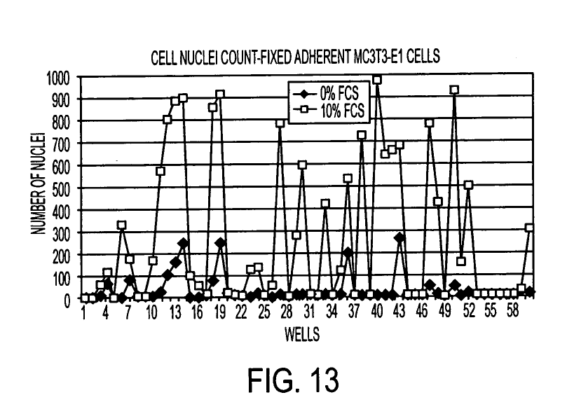

An image analysis software package (Meta Morph, Universal Imaging Corporation, a subsidiary of Molecular Devices, Downingtown, Pa.) was used to enumerate the fluorescently labeled cell nuclei in FIG. 12 and the nuclei count results for both cells in media containing no fetal calf serum and media containing 10% fetal calf serum are shown in FIG. 13. In FIG. 13, wells 1-10 correspond to row B, columns 2-11 in FIG. 9; wells 11-20 in FIG. 12 correspond to row C, columns 2-11 in FIG. 12, etc.

In FIG. 13, in the presence of 10% fetal calf serum, cell adhesion was observed for a number of wells. In the absence of serum, cell adhesion was reduced, but cell adhesion was still observed in a number of wells. In both cases, cell adhesion in some wells containing cell adhesion ligands according to the statistically designed experiment exceeded that of cells cultured on plain tissue-culture grade polystyrene (wells 59 and 60 in FIG. 12). The results obtained enabled the identification of a number of surfaces that support MC3T3-E1 adhesion better than tissue culture grade polystyrene, the most commonly used cell culture support.

In order to optimize the surfaces, one can follow two leads, e.g., the “best well” composition or the “best factors”. The determination of “best factors” is made following rigorous statistical analysis of the experimental results.

In the “best well” approach, the well with the best experimental outcome is chosen for further optimization. In the example shown in FIG. 13, one would choose well 40 (or well E11 ) which had the highest number of cell nuclei. This well contained a mixture of Collagen-type VI and Collagen-type III according to the plate layout shown in FIG. 11. The concentration of Collagen-type VI and Collagen-type III that was chosen for the immobilization step in the ECM screening plate preparation was based on initial concentration-dependent studies with the MC3T3-E1 cells using the model ECM, fibronectin. It is noted that a concentration which is optimal for one cell-type under investigation may not be optimal for another cell-type. Moreover, the concentration of a particular ECM which is optimal for a given cell-type may not be the optimal concentration for another ECM, even when the same cell type is used. Similarly, the composition of a mixture in the “hit well” may not be optimal. For example, the surface of well E11, which was the “best well” comprised a 50/50 mixture of Collagen-type VI and Collagen-type III. Follow-up experiments may be performed to optimize the concentration of both ligands chosen for the immobilization step, as well as the composition of the mixture (a 50/50 mixture may not be the optimal composition) bound to the surface of a “hit” well for a given cell-type.

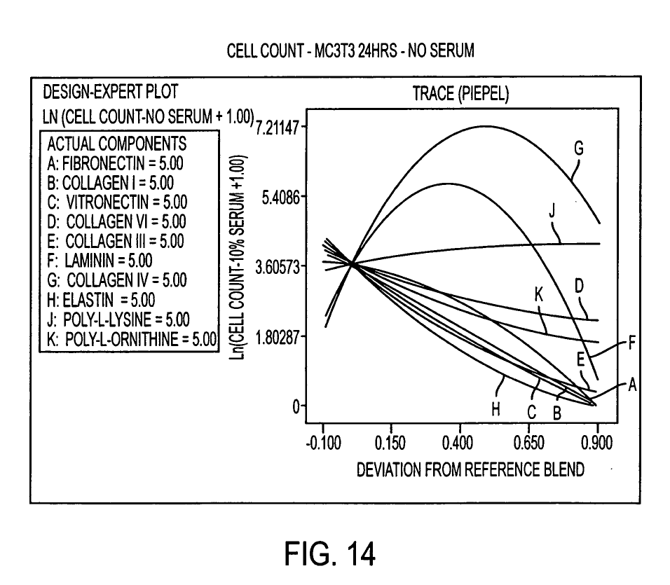

In the “best factors” approach, the experimental results are analyzed using statistical models. For the above-described example, a mixture-model analysis of the MC3T3-E1 data shows that Collagen IV, laminin, and poly-L-lysine (marginal effect) appear to increase the cell count when present at significant quantities with no serum as shown in FIG. 14. The points at which all the lines intersect correspond to mid-points, where all 10 ECMs were present at 5 μL each. This graph provides an indication as to how the cell count changes, depending on how far the well composition deviates from this reference “mid-point” blend. As can be seen, as the amount of Collagen IV or laminin increases, the cell counts increase.

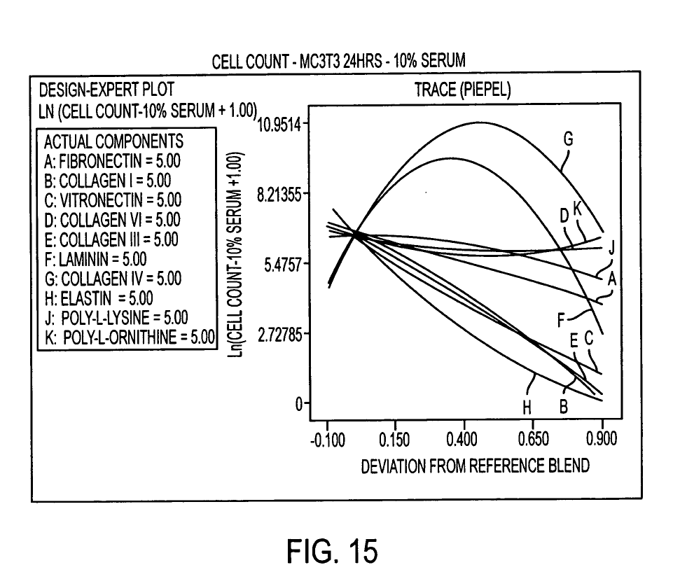

With reference now to FIG. 15, with 10% serum, any effect of poly-L-lysine that was seen in FIG. 14 diminishes, and only Collagen IV and laminin continue to show a positive effect on cell count.

It is noted that both the “best well” and “best factors” approaches are valid, but each approach can lead to different surface compositions. In the present example, the “best well” approach would lead to a surface comprising Collagen-type VI and Collagen-type III, while the “best factor” approach would lead to a surface comprising Collagen VI and laminin.

### Example 5

### Use of a Statistically Designed Experiment (Plackett-Burman Design) to Screen 30 Different Agents

Design

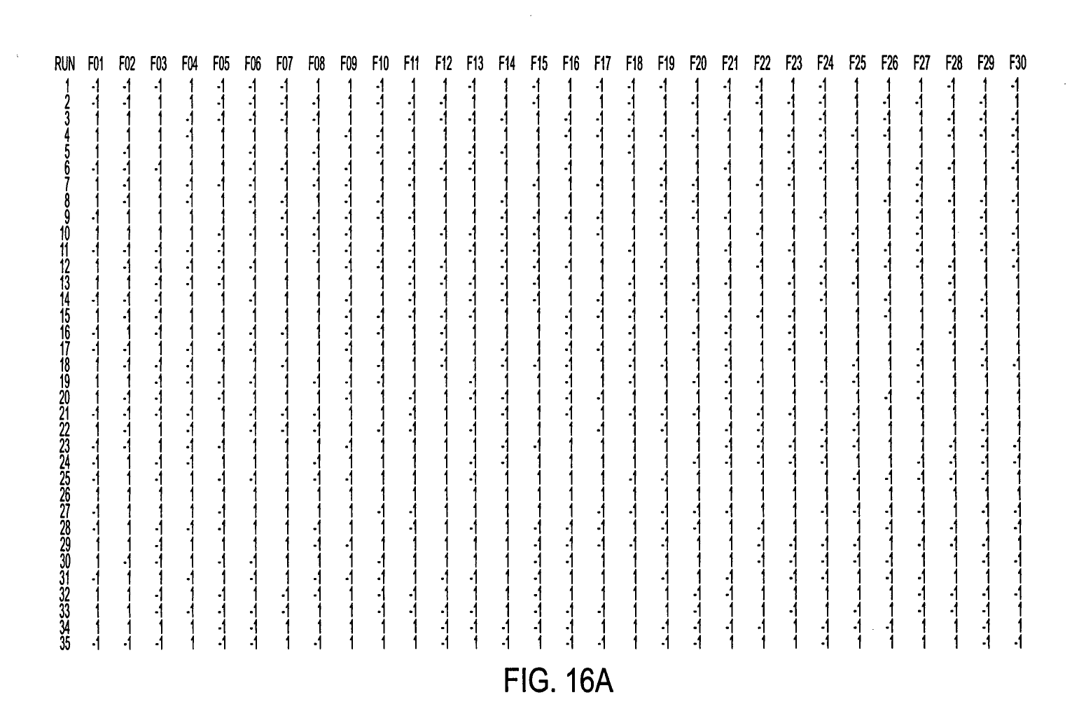

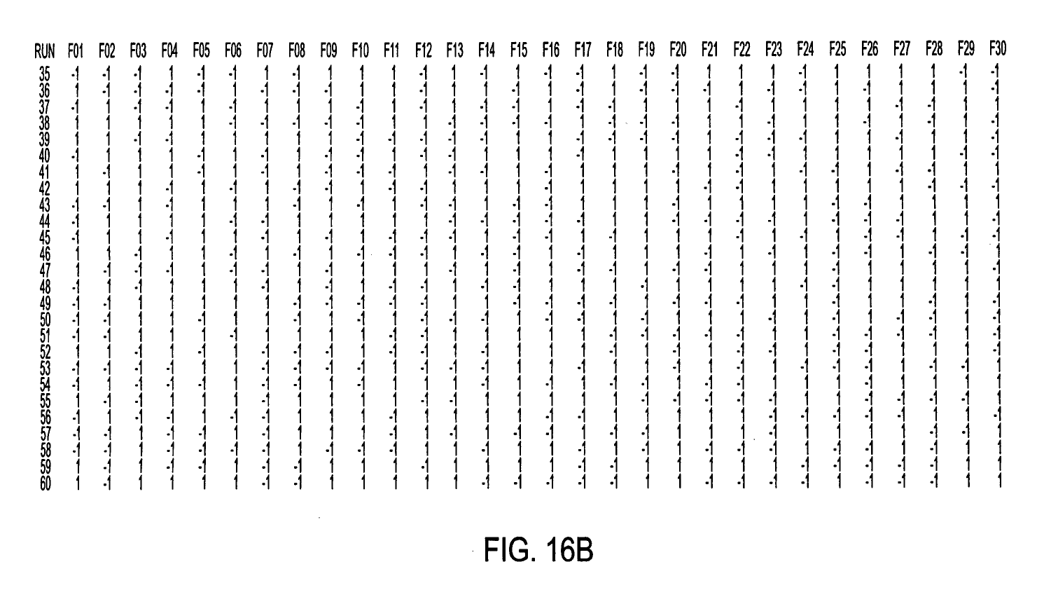

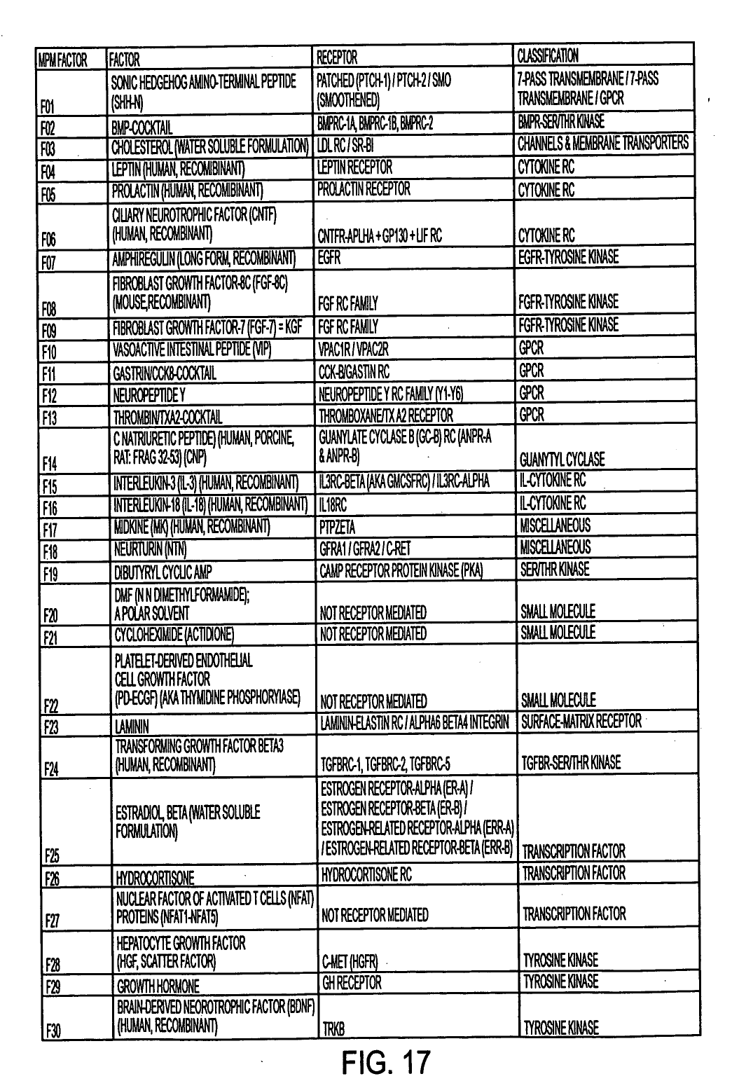

The present example describes a Plackett-Burman (PB) design as shown in FIG. 16 (a-d), which was generated using a commercially available software package JMP™ from SAS Institute (Cary, N.C.). In particular, the screening design was generated using the custom design function in SAS/JMP V 4.0.5. The software package is a GUI oriented package, so there is no code to show. With reference to FIG. 16a, the first column is a list of the experimental points (runs) that translate into single wells in the 96-well plate, e.g., 60 wells in this case. The numbers in the spreadsheet itself (−1 or 1) (FIGS. 16a-d) is an indication of the level of a factor. In this example, “1” indicates the presence of the factor and “−1” indicates the absence of a factor. Moreover, in this example, if a factor is present in a given well, it is always at the same concentration in regard to the total volume of the well. The total concentration of agents may vary from well to well based on the number of agents included in the corresponding experimental run. The generic factor names are provided in the top row of FIGS. 16 a-d. FIG. 17 shows the identity of each of generic factors F01-F30 in the present experiment. For example, experimental run 1 in the first column may represent well 1 of a 96-well plate. From the statistical design shown in FIG. 16 (a-d), it can be seen that the following factors are present (i.e., level “1”) in well 1: F04, F08, F09, F11, F12, F14, F16, F20, F23, F25, F26, F27, and F29.

Proposed Acquisition of Data and Statistical Analysis

Cells are plated into the wells of a 96-well plate in accordance with the design shown in the spreadsheet of FIG. 16 (a-d). The seeding density is about 10,000 cells per well. Cells are incubated on the plates overnight at 37° C. The following day, media and any cells not adhering to the immobilized agents on the well surfaces are removed and any adhered cells are fixed by exposure to formalin for 15 minutes. The nuclei of the fixed adhered cells are fluorescently labeled and images are acquired with a fluorescent microscope as described above in Example 4. An image analysis software package (Meta Morph, Universal Imaging Corporation) is used to enumerate the fluorescently labeled cell nuclei and the nuclei count results for the cells are obtained. Based on these results, wells with the best experimental outcome (e.g., highest number of cell nuclei) are chosen for further optimization. By examining the contents of the wells that give the best results, information is gained regarding which factors and/or factor groups yields beneficial effects. By including many factors in the design, potentially more complex interactions between the factors can be determined. Follow-up screening experiments can focus on a particularly interesting factor combination discovered in the first round of screening.

Following the first screen, the main effects are estimated and reviewed. By “main effects”, it is meant the effect of a single agent acting independently. Interaction effects mean the combined effects of more than one single agent when the agents act in concert (not independently). At this point, relevant interactions among the agents typically are not estimated in the statistical model, but interactions among the agents would be expected to result in the best experimental runs, i.e., best wells. After the first round of screening, the best wells and the factors that are included in these wells (level=“1”) are identified. Follow-up experiments can be performed for each best well using all the factors included in the well, whether or not they had a positive, neutral, or negative effect in the preliminary statistical analysis. The experiments can be repeated with a subset of the agents identified in the best well so as to arrive at an optimum subset of factors for producing a desired response in a cell. Moreover, the experiment can be repeated, wherein the concentration of the agents in a best well are varied. Follow-up experiments can also be performed with the subset of single agents that had statistically significant main effects or by combining a subset of the best single agents with a subset identified in the best mixtures.

It has been proposed that the control of cellular phenotypes via extracellular conditions is governed by high order interactions among the factors in the extracellular environment. The Plackett-Burman design presented here is believed to provide good statistical estimates of the main effects and also provides the opportunity to observe a diverse set of combinations of factors among its experimental runs. In this case, higher-order interactions would be expected to result in specific experimental runs being “best wells” over and above what could be predicted by the individual main effects of the agents in the best wells.

Although only a few exemplary embodiments of the present invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims.

1. An automated method for providing an optimization strategy to identify the optimum concentration of a plurality of substances that optimizes cell culture conditions based upon a variety of responses, the method comprising the steps of:
generating a statistical design that maps each of a plurality of substances to a respective generic factor name;
generating said statistical design that further maps at least one concentration of each of said substances to a respective factor level;
generating said statistical design that further maps respective locations of a plurality of receptacles in a first receptacle array;
generating a first representation according to said statistical design and in response, placing a respective concentration of one of said substances and a respective concentration of another of said substances as a respective combination substance into at least one of said receptacles in said first receptacle array;
contacting said placed combination substance with cells of interest; and
acquiring data indicative of a phenotypic change in said cells of interest due to contact between said cells of interest and said combination substance and in response, determining an optimum combination substance and an optimum concentration of said substances of said optimum combination substance resulting in a desired phenotypic change.

2. A method for providing an optimization strategy as claimed in claim 1, wherein:
said placing includes placing respective concentrations of a respective two of said substances as respective combination substances into respective said receptacles;
said contacting includes contacting said cells of interest with each of said combination substances in said receptacles; and
said acquiring includes acquiring respective said data indicative of a respective phenotypic change in said cells of interest due to contact between said cells of interest and said respective combination substances and in response, determining said optimum combination substance and said optimum concentration of said substances of said optimum combination substance resulting in said desired phenotypic change.

3. A method for providing an optimization strategy as claimed in claim 1, wherein said combination substance comprises one of the following:
said respective concentration of said one of said substances having a first concentration value combined with said respective concentration of said another of said substances having a second concentration value; and
said respective concentration of said one of said substances having a third concentration value combined with said respective concentration of said another of said substances having a fourth concentration value.

4. A method for providing an optimization strategy as claimed in claim 3, wherein:
said first concentration value of said one of said substances is less than or equal to said third concentration value of said one of said substances; and
said second concentration value of said another of said substances is less than or equal to said fourth concentration value of said another of said substances.

5. A method for providing an optimization strategy as claimed in claim 1, further comprising the steps of:
generating said statistical design that further maps respective locations of a plurality of receptacles in a second receptacle array;
generating a second representation according to said statistical design and in response, placing a respective concentration of one of said substances, a respective concentration of another of said substances and a respective concentration of a further one of said substances as a respective three-combination substance into at least one of said receptacles in said second receptacle array;
contacting said placed three-way combination substance with cells of interest; and
acquiring second data indicative of a phenotypic change in said cells of interest due to contact between said cells of interest and said three-way combination substance and in response, determining an optimum three-way combination substance and an optimum concentration of said substances of said optimum three-way combination substance resulting in a second desired phenotypic change.

6. A method for providing an optimization strategy as claimed in claim 5, wherein:
said placing includes placing respective concentrations of a respective three of said substances as respective three-way combination substances into respective said receptacles of said second receptacle array;
said contacting includes contacting said cells of interest with each of said three-way combination substances in said receptacles of said second receptacle array; and
said acquiring includes acquiring respective said second data indicative of a respective phenotypic change in said cells of interest due to contact between said cells of interest and said respective three-way combination substances and in response, determining said optimum three-way combination substance and said optimum concentration of said substances of said optimum three-way combination substance resulting in said second desired phenotypic change.

7. A method for providing an optimization strategy as claimed in claim 6, wherein said three-way combination comprises:
said respective concentration of said one of said substances having a first concentration value combined with said respective concentration of said another of said substances having a second concentration value and said respective concentration of said further of said substances having a third concentration value.

8. A method for providing an optimization strategy as claimed in claim 5, further comprising the steps of:
generating said statistical design that further maps respective locations of a plurality of receptacles in a third receptacle array;
generating a third representation according to said statistical design and in response, placing said optimum combination substance in at least one receptacle in said third receptacle array and placing said optimum three-way combination substance into at least one other receptacle in said third array;
contacting said placed optimum combination substance and said placed optimum three-way combination substance with cells of interest; and
acquiring third data indicative of a phenotypic change in said cells of interest due to contact between said cells of interest and said optimum combination substance and said optimum three-way combination substance and in response, determining an optimum concentration of said substances of said optimum combination substance and said optimum three-way combination substance resulting in a third desired phenotypic change.

9. A method for providing an optimization strategy as claimed in claim 8, wherein:
said placing includes placing said optimum combination substance in a plurality of said receptacles and placing said optimum three-way combination substance in a plurality of other of said receptacles in said third array.

10. A method for providing an optimization strategy as claimed in claim 8, wherein:
said placing further includes placing at least one other three-way combination substance in other of said receptacles in said third array;
said contacting further includes contacting said placed one other three-way combination substance with said cells of interest; and
said acquiring includes acquiring said third data indicative of a phenotypic change in said cells of interest due to contact between said cells of interest and said optimum combination substance, said optimum three-way combination substance and said one other three-way combination substance and in response, determining an optimum concentration of said substances of said optimum combination substance, said optimum three-way combination substance and said one other three-way combination substance resulting in said third desired phenotypic change.

11. An automated method for providing an optimization strategy to identify the best concentration of a plurality of substances that optimizes cell culture conditions based upon a variety of responses, the method comprising the steps of:
generating a statistical design that maps each of a plurality of substances to a respective generic factor name;
generating said statistical design that further maps at least one concentration of each of said substances to a respective factor level;
generating said statistical design that further maps respective locations of a plurality of receptacles in each of a first, second and third receptacle array;
determining an optimum combination substance based on a response of said cells of interest placed in contact with respective two-way combinations of said substances in said receptacles in said first receptacle array;
determining an optimum three-way combination substance based on a response of said cells of interest placed in contact with respective three-way combinations of said substances in said receptacles in said second receptacle array; and
determining an optimum substance concentration based on a response of said cells of interest placed in contact with said optimum combination substance in certain of said receptacles in said third receptacle array and said optimum three-way combination substance in certain other of said receptacles in said third receptacle array.

12. A method for providing an optimization strategy as claimed in claim 11, wherein:
said determining steps determine said optimum combination substance, said optimum three-way combination substance and said optimum substance concentration based on observed respective phenotypic changes of said cells of interest in said receptacles of said first, second and third receptacle arrays.

13. A method for providing an optimization strategy as claimed in claim 11, wherein:
said two-way combinations each include respective concentrations of each of two of said substances, and said three-way combinations each include respective concentrations of each of three of said substances.

14. A method for providing optimization strategy as claimed in claim 13, wherein:
said respective concentrations of said two of said substances in said two-way combinations include one of the following:
a respective first concentration value of one of said two substances and a respective second concentration value of the other of said two substances; and
a respective third concentration value of one of said two substances and a respective fourth concentration value of the other of said two substances; and
said respective concentrations of said three of said substances in said three-way combinations include a respective fifth concentration value of each of said three substances.

15. A method for providing optimization strategy as claimed in claim 14, wherein:
said first concentration value of said one of said two substances is less than or equal to said third concentration value of said one of said two substances; and
said second concentration value of said other of said two substances is less than or equal to said fourth concentration value of said other of said two substances.

16. An automated method for providing an optimization strategy to identify the optimum concentration of a plurality of substances based upon a variety of responses, the method comprising the steps of:
identifying at least one two substance combination subset for inclusion in a subset optimum concentration evaluation using a first statistically created plate layout;
identifying at least one three substance combination subset for inclusion in a subset optimum concentration evaluation using a second statistically created plate layout; and
identifying at least one optimum substance concentration from said identified said two substance combination subset and said three substance combination subset using a third statistically created optimization plate layout.

17. An automated method for providing an optimization strategy as claimed in claim 16, further comprising:
said identifying at least one two substance combination subset from a plurality of substances in which a first and second substance is combined and at least one substance concentration level is varied between a high and low concentration level.

18. An automated method for providing an optimization strategy as claimed in claim 16, further comprising:
said identifying at least one three substance combination subset from a plurality of substances in which a first, second and third substance is combined.

19. An automated method for providing an optimization strategy as claimed in claim 16, further comprising:
said identifying at least one optimum substance concentration from said two substance combination subset and said three substance combination subset in which at least one substance concentration level is varied between a plurality of substance concentration levels.

20. An automated method for providing an optimization strategy as claimed in claim 16, further comprising:
said identifying at least one optimum substance concentration from said two substance combination subset and said three substance combination subset based on data indicative of a phenotypic change in cells of interest when placed in contact with said two substance combination subset and said three substance combination subset.
